(19) United States
(12) Patent Application Publication
(10) Pub. No.: US 2022/0098237 A1
(43) Pub. Date: Mar. 31, 2022

(54) COMPOUND, SYNTHESIS INTERMEDIATE, USE, PHARMACEUTICAL COMPOSITION AND NEUROMODULATORY THERAPEUTIC METHOD

(71) Applicants: Remer Consultores Assessoria Empresarial Ltda, Rio de Janeiro (BR); Proteinmax Biotecnologia Ltda, Butantã (BR)

(72) Inventors: Ricardo Amaral Remer, Rio de Janeiro (BR); Andrea Sterman Heimann, São Paulo (BR)

(21) Appl. No.: 16/612,220

(22) PCT Filed: May 14, 2018
(86) PCT No.: PCT/BR2018/050156

§ 371 Date: Oct. 12, 2021

(30) Foreign Application Priority Data
May 15, 2017 (BR) 102017010169-0
Oct. 11, 2017 (BR) PCT/BR2017/050314

(51) Int. Cl. C07K 5/113; C07K 5/093; C07K 5/103; A61P 29/00; A61P 25/08; A61P 25/28

(52) U.S. Cl. CPC C07K 5/1021; C07K 5/0819; C07K 5/101; A61K 38/00; A61P 25/08; A61P 25/28; A61P 29/00

(57) ABSTRACT

A peptide compound in the fields of pharmaceutical sciences, medicine, chemistry and biotechnology, that shows stability and ease of handling when compared to the more closely related peptidic compounds. A pharmaceutical composition that includes the peptidic compound and shows therapeutic results even when administered orally. In some embodiments, administration of the pharmaceutical composition provides superior therapeutic results when compared to the effects of hemopressin and cannabidiol. In some embodiments, oral administration of the pharmaceutical composition provides neuromodulation, both in the curative or prophylactic treatment of seizures, modulation of pain threshold and important neuroprotection, and reduction of the clinical symptoms of Multiple Sclerosis.

### CROSS REFERENCE TO RELATED APPLICATIONS

This patent application is the US National Phase of International Application No. PCT/BR2018/050156 having an International Filing Date of 14 May 2018, which claims priority on International Application No. PCT/BR2017/050314 having an International Filing Date of 11 Oct. 2017 and Brazilian Patent Application No. 102017010169-0 having a filing date of 15 May 2017.

A Sequence Listing in the format ASCII is herein by incorporated. The name of the ASCII text file is “RemerProteimax PCT Neuro2 Sequence listing” created on 11 Oct. 2021, and the size of the ASCII text file is 1519 bytes.

### BACKGROUND OF THE INVENTION

The present invention is in the fields of pharmaceutical sciences, medicine, chemistry and biotechnology. More specifically, the present invention describes a compound and its use for preparing a ligand of diagnostic and/or therapeutic interest, a synthetic intermediate in the preparation of compounds of pharmaceutical interest, the use of a compound for preparing a medicament, a pharmaceutical composition containing said compound and a therapeutic method. The compound of the present invention is peptidic and showed surprising stability and ease of handling when compared to the more closely related peptidic compounds. The pharmaceutical composition of the invention comprises said peptidic compound and showed surprising therapeutic results even when administered orally. In some embodiments, administration of the composition of the invention provided superior therapeutic results when compared to the effects of hemopressin and cannabidiol. In some embodiments, oral administration of the composition of the invention provides important and surprising results in the curative or prophylactic treatment of seizures, modulation of pain threshold and important neuroprotection, having substantially reduced the clinical symptoms of Multiple Sclerosis.

The peptidic compound of the invention is surprisingly stable at extreme temperatures, does not pose the problem of fibril formation and provides for ease of handling in the preparation of products of pharmaceutical interest, including ligands for diagnostics and pharmaceutical compositions.

The peptidic compound which is closest to the present invention is hemopressin and its larger size variants with biological activity. However, hemopressin presents technical difficulties such as instability and tendency to form fibrils, which limit its use in pharmaceutical preparations (Bomar, M. G. and Galande, A. K., Modulation of the cannabinoid receptors by hemopressin peptides, 520-524). These limitations have diminished initial enthusiasm with hemopressin: its natural tendency for aggregation/fibril formation substantially limits the range of possible concentrations at both the synthesis, pharmaceutical preparation and therapeutic use. The same literature (Bomar and Galande) goes so far as to speculate that hemopressin self-assembly may be physiologically relevant and potentially pathogenic or neurotoxic, analogously to the amyloid peptidic fibrils associated with Alzheimer's disease, Parkinson's disease and type II diabetes.

The results of the present invention are surprising in view of the closest background, and the present inventors are not aware of any report or suggestion in the literature that the peptidic compound of the invention would be more stable and more easily manipulable than Hemopressin, thus being in relation to this, an unexpected improvement and of unexpected magnitude.

The cannabinoid system, which comprises the CB1 and CB2 receptors and their endogenous ligands, acts on various metabolic functions, including control of food intake, energy and/or lipid metabolism, regulation of intestinal motility, immune system, in the balance of the calcium cycle, among others. Cannabinoid receptors are widely expressed in the brain, including cortex, hippocampus, amygdala, pituitary, and hypothalamus. CB receptors, particularly CB1, have already been identified in numerous peripheral organs and tissues, including the thyroid gland, adrenal gland, reproductive organs, adipose tissue, liver, muscle, and gastrointestinal tract.

Bomar and Galande's article also shows that N-extended versions of hemopressin, such as RVD-Hp, VD-Hp, have shown an agonist effect on the CB1 receptor, and that, therefore, a difference of only two or three amino acids appears to interfere if the peptide exhibits antagonistic or agonist effect, so as to produce opposite effects on CB1. In addition, the signaling pathways for the different known peptides are different from the classical pathway mediated by the G protein, so that no extrapolation is reliable in light of the literature reports available so far. Similar conflicting effects may also occur with respect to the CB2 receptor.

The literature also clearly shows that while hemopressin and said known extended-size variants have therapeutic activity, peptidic compounds of size less than six amino acids do not provide therapeutic effect (Szlavicz et al., 2015; Dvoracsko et al., 2016; Bomar and Galande, 2013; Bauer et al., 2012).

The present invention surprisingly proves the opposite: in addition to providing more intense therapeutic effects in the known uses for hemopressin and extended-size variants, it also provides other uses not previously known or suggested. In addition, small changes in peptide sizes may not only lead to substantial and unexpected changes in therapeutic effects, but may also lead to the complete absence of effects.

It should also be noted that studies with peptides resembling hemopressin (pepcans) have been facing several challenges. For example, manipulation of said larger peptides has been hindered by fibril aggregation/formation. In addition, the metabolic stability of said peptides in vivo is limited and data from the respective pharmacological experiments are difficult to interpret without pharmacokinetic analysis, leading to confusion in the literature. Although there are studies with hemopressin (Hp) and RV-Hp administration, the only endogenous peptides naturally present at physiological levels appear to be RVD-Hp (pepcan 12) and pepcan 23.

In this context, various compounds have already been detected in the art as having cannabinoid receptor modulation activity. Among these, some target the development of drugs for weight reduction and thinning of the waist, such as rimonabant. However, this compound was subsequently associated with increased occurrence of psychiatric disorders in humans and was removed from the world market.

The compound of the invention additionally provides the advantage of being a good candidate as substitute to the cannabinoid compounds, such as cannabidiol. Cannabidiol, despite its proven effects, has been facing regulatory problems due to its origin, the Cannabis sativa plant. The present invention provides an additional therapeutic approach for patients suffering from seizures and having difficulty obtaining drugs, being based on a peptidic compound and not using derivatives of Cannabis sativa. The results showed that the compound of the invention, when used as an anticonvulsant, provides other surprising technical advantages in use, including increased therapeutic effect, oral use, lower dosage, less occurrence of side effects such as prostration and nasal bleeding, among other technical advantages.

Pilocarpine (commonly called “Pilo”) is an alkaloid extracted from the leaves of the jaborandi plant (Pilocarpus jaborandi), a plant used for centuries by the Tupi-Guarani Indians who inhabit Brazil and take advantage of their properties to produce sweat and saliva. Pilocarpine is a nonspecific muscarinic agonist, slowly degraded and without effects on nicotinic receptors and was introduced in clinical practice by the Brazilian physician Sifronio Coutinho, in 1874, through extracts from the jaborandi leaf to obtain a diaphoretic (sweat production) and silagogue (production of saliva) effect.

Despite its pharmaceutical properties, pilocarpine at high concentrations induces the occurrence of convulsions, being used as an experimental model therefor. Pilocarpine-induced seizures lead to neurotoxicity at the cellular level and may be related to increased cerebral oxidative stress and changes in the concentration of certain amino acids (Santos et al., 2011).

Administration of pilocarpine in this experimental model leads to severe brain injury, neurotoxicity and usually culminates in the death of animals. However, before this outcome, pilocarpine causes cholinergic alterations capable of inducing status epilepticus (SE) associated with convulsive stereotyped movements. Pilocarpine is able to induce status epilepticus either administered directly into the brain or intraperitoneally. This substance was used in some experiments described in this patent application, its harmful neuronal/encephalic effects having been inhibited or prevented by the composition of the present invention: substantial part of the animals subjected to these experiments had no symptoms related to brain lesions and survived without apparent damage, in contrast to groups of animals treated with other known substances.

Much of the understanding of the mechanisms of epilepsy comes from studies in animal experimental models, especially in rats and mice. In this context, the administration of pilocarpine in rodents mimics epilepsy (ELT) in humans and is generally referred to as the “pilocarpine model”. This model was developed in 1983 by Turski et al. and is now one of the most widely used models of epilepsy, since its histological, biochemical, pharmacological, electrophysiological and behavioral characteristics (Turski et al., 1983) similarly reproduce those found in human carriers of ELT.

The pilocarpine model is also useful for evidencing changes in muscarinic receptors, such as the occurrence of salivation. Acetylcholine, through its muscarinic receptor (mAChRs) plays an important role in cognitive functions, such as learning and memory. mAChRs are receptors that form complexes with G protein-receptor in the cell membranes of certain neurons and other cells. They play several roles, including acting as the ultimate end receptor stimulated by acetylcholine released from postganglionic fibers in the parasympathetic nervous system.

Muscarinic receptors are so-called because they are more sensitive to muscarin than to nicotine. Its counterparts are nicotinic acetylcholine receptors (nAChRs), ion channels receptors that are also important in the autonomous nervous system. Many drugs and other substances (e.g., pilocarpine and escopolamine) manipulate these two distinct receptors acting as selective agonists or antagonists.

The mAChRs are among the most well characterized among the transmembrane receptors (7TM), being widely expressed in the central nervous system (CNS). Five mAChR subtypes were cloned (M1, M2, M3, M4 and M5) and are generally divided into two distinct classes based on signal transduction. mAChR M1, M3 and M5 are subtypes that signal through Gq/11 proteins and activate phospholipase-C and mobilize intracellular calcium. mAChR M2 and M4, however, predominate through Gi/o proteins inhibiting adenylate cyclase and reducing the intracellular concentration of cAMP. The predominant mAChR in the CNS is the M1 subtype, which is located in the cortex, hippocampus, striatum and thalamus, where it is found post-synaptic. M2 mAChRs are located predominantly in the brainstem and thalamus, although also in the cortex, hippocampus and striatum, where they control the release of acetylcholine. mAChRs of M3 and M5 are expressed at much lower levels than M1 or M2 mAChRs in the CNS, but M3 mAChRs are found in the cortex and hippocampus, while M5 mAChRs have a very discrete location in the substantia nigra. M4 mAChRs are found in many regions of the brain, including the cortex and hippocampus, but are more prominent in the striatum, where they are thought to play a role in the control of dopamine release and modulate locomotor activity.

Given the broad and varied expression profile of mAChRs in the CNS, it is not surprising that all subtypes have been evaluated as potential drug targets. Some of these targets are well accepted in the literature, such as the involvement of M1 in Alzheimer's disease, while others are relatively new, such as M5 that has been correlated with dependency and addiction to abuse drugs.

Multiple sclerosis is a neuroinflammatory disease of the central nervous system with a strong neurodegenerative component, and has a great impact on the health of the patients. The disease still has few satisfactory available therapeutic options, and the development of alternatives is highly desirable, as is the case of the present invention.

Multiple sclerosis is characterized by the destruction of oligodendrocytes and neurons, resulting in heterogeneous and cumulative clinical symptoms. The currently available therapies are only partially effective and are primarily directed to the inflammatory phase of the disease. However, the neurodegenerative component of the disease is still the greatest challenge for new therapeutic approaches. The present invention is precisely intended to fill this important gap.

Although the exact etiology of the disease is still unclear, it is speculated that self-reactive T lymphocytes play a central role in its pathophysiology. The most common animal model of Multiple Sclerosis is experimental autoimmune encephalomyelitis (EAE), because it shares many physiological and clinical aspects. There are several models of EAE that reflect different clinical, immunological and histological aspects of human multiple sclerosis. The active induction model of EAE in mice is robust and provides replicable results, being particularly useful in the search for therapeutic agents through the use of transgenic mice challenged by autoimmune neuroinflammation. The EAE model is often used as a ‘proof of principle’ of the efficacy of novel therapeutic strategies for Multiple Sclerosis.

Background searches have disclosed documents only partially relevant for the present invention. Such documents will be described below, being set forth herein solely for the purpose of serving as a basis for the state of the art, since none of them anticipates or even suggests any of the objects of the present invention.

Some studies presented by one of the present inventors in Novel Peptide Substrates for Endopeptidase 24.15 Neurolysin and Angiotensin Converting Enzyme (Vanessa Rioli, Fabio C. Gozzo, Andrea S. Heimann, Alessandra Linardi, Jose E. Krieger, Claudio S. Shida, Paulo C. Almeida, Stephen Hyslop, Marcos N. Eberlin, Emer S. Ferro; Mar. 7, 2003) demonstrate an effective technique of “screening” new peptides. The present invention differs from that document, among other reasons, because it provides a different molecular entity. It also provides a more stable and more therapeutically effective compound. In addition, it provides a novel therapeutic approach to neuromodulation, neuroprotection, analgesia, convulsions or the treatment of multiple sclerosis, the results of which have been surprising.

Gomes et al. (Gomes I, Grushko J S, Golebiewska U, Hoogendoorn S, Gupta A, Heimann A S, Ferro E S, Scarlata S, Fricker L D, and Devi L A Novel endogenous peptide agonists of cannabinoid receptors FASEB J. 2009 September; 23 (9): 3020-3029) is another scientific publication which has as one of the authors the co-inventor in the present invention (Heimann). Said co-inventor, when tests were proposed by the other co-inventor in order to verify the hypothesis of the present invention, was surprised by the hypothesis—which itself reveals the non-obviousness thereof. It should be noted that Gomes et al. was published in 2009, while the priority of the present invention is 2017, i.e., even more than seven years after the publication of Gomes et al. and practically ten years after the discovery of hemopressin (Hp) by the inventor herself (Heimann et al., Proc Natl Acad Sci USA, 2007 Dec. 18; 104 (51): 20588-93), not only she but no other researcher in the world had published or suggested the objects of the present invention.

Furthermore, Gomes et al. disclose the natural presence of certain endocannabinoids in the brain of mice (i.e., local expression) having found N-extended versions of Hp. In addition, one of the conclusions of Gomes et al. is that the N-extended versions found in the brain (RVD-Hpα and VD-Hpα) have opposite function to that of the peptide of the present invention, this being antagonist/inverse agonist and those being agonist of cannabinoid receptors. Therefore, Gomes et al. neither disclose or suggest the use of any endocannabinoid in the control of seizures or on neuromodulation, nor pharmaceutical compositions comprising any of these entities—much less the use of the peptide of the invention, any of the claimed uses, or oral compositions comprising the same. Consequently, not only Gomes et al. do not disclose the use of the peptide of the invention, but the person skilled in the art, from reading Gomes et al., would not be motivated to: (i) synthesize the peptidic compound of the invention; (ii) expect its stability to be substantially greater than that of Hemopressin; (iii) expect the therapeutic action to be greater than that of Hemopressin; (iv) test it for the other uses indicated herein with reasonable expectation of success; or (v) prepare oral pharmaceutical compositions for neuromodulation, neuroprotection, seizure or multiple sclerosis treatment. In this context, the co-inventor herself did not see much sense in such approach before performing the tests suggested by the other co-inventor. The compound of the invention is novel and has surprising properties; in previously known uses, is a substantial and surprising improvement; in previously unknown uses, it is novel and surprising, as it is also surprising that an oral pharmaceutical composition containing a peptide has a neuromodulatory action. One skilled in the art would not expect said peptidic compound of the invention to have greater stability and resist the gastrointestinal tract and interact in order to produce obtained the neuromodulation/neuroprotection effects.

Document WO 2014/008567 has as one of the inventors the co-inventor in the present invention (Heimann). It discloses peptidic compounds and compositions useful for treating metabolic disorders comprising obesity, diabetes, systemic arterial hypertension (or disease, condition related and/or associated comorbidities); prevention of overweight; regulation of appetite; induction of satiety; prevention of weight gain after successful weight loss; increased energy consumption; aesthetic weight reduction; or bulimia. The compounds of said document interact with CB receptors, but are of different chemical structure than those of the present invention. Furthermore, no report or suggestion of use of the compound of the invention as a neuromodulator, neuroprotectant, anticonvulsant or as an entity useful in the treatment of multiple sclerosis is made in said document, because this approach was neither imagined by the inventors nor was it obvious from the state of the art.

Document WO 2011/011847 (=US2015/297669, U.S. Pat. No. 9,452,196, EP2459207) has as one of the inventors the inventor of the present invention (Heimann). This document only discloses the use of hemopressin (Hp) for the control of diabetes and obesity/weight gain and is limited to Hp and larger derivatives such as DV-Hp, PVD-Hp, RVD-Hp. The present invention differs from said document, among other reasons, for disclosing a different compound, which provides benefits when compared to hemopressin, including more ease of preparation, greater stability and biological activity, among other advantages. In addition, the compound of the invention provides other therapeutic uses not disclosed or suggested in WO 2011/011847: the test performed by the present inventors showed surprising results, especially in regard to neuromodulation, neuroprotection, inhibition of seizures, and of multiple sclerosis symptoms, facts not described or suggested in said document. In addition, said co-inventor of WO 2011/011847, when the other co-inventor proposed tests to verify the hypothesis of the present invention, was struck by the hypothesis—which in itself reveals the non-obviousness thereof. Document WO 2011/011847 was published in 2011, while the priority of the present invention is 2017, i.e., even more than five years after the publication of the earliest precedent and practically ten years after the discovery of hemopressin by the co-inventor herself (Heimann et al., Proc Natl Acad Sci USA, 2007 Dec. 18; 104 (51): 20588-93), not only she but no other researcher in the world had published or suggested the objects of the present invention. Finally, document WO 2011/011847 focuses on the action on receptors present in adipose tissue, which are outside the brain and not in brain receptors.

It is also important to note that the literature makes no mention to hemopressin, much less the peptide of the present invention, for the control of seizures. The use of hemopressin in seizure control has not been disclosed or suggested in the prior art and is the subject matter of another co-pending application of the same inventors, PCT/BR2017/050313. What the literature reveals are other molecular entities and which are CB1 receptor agonists, while the peptide of the invention is an inverse agonist and antagonist (modulator), which makes the results of the present invention even more surprising.

Document WO 2013/021196 discloses the use of hemopressin as an agent that interferes with the differentiation of oligodentrites. The present invention differs from said document, among other reasons, in that it reveals other compounds, other uses and provides benefits when compared to hemopressin, including more ease of preparation, greater stability and biological activity, among other advantages. In addition, the tests performed by the inventors showed surprising results, particularly in regard to neuromodulation, neuroprotection, inhibition of the occurrence of seizures and/or symptoms of multiple sclerosis with the compounds of the invention, which are neither described nor suggested in said document.

Other references of scientific literature which circumscribe the invention, but without anticipating or suggesting it, include the following documents.
Asproni, B. et al., Novel pyrrolocycloalkylpyrazole analogues as CB1 ligands. Chemical Biology and Drug Design, p. 1-13, 2017.
Hildebrandt, A. K. et al., Efficient computation of root mean square deviations under rigid transformations. Journal of Computational Chemistry, v. 35, p. 765-771, 2014.
Hua T. et al., Crystal structure of the human cannabinoid receptor CB1. Cell, v. 167, p. 750-762, 2016.
Maiorov, V. N.; Crippen, G. M., Significance of root-mean-square deviation in comparing three-dimensional structures of globular proteins. Journal of Molecular Biology, v. 235, p. 625-634, 1994.
Morgan, C. A.; Hurley, T. D., Characterization of two distinct structural classes of selective aldehyde dehydrogenase 1A1 inhibitors. Journal of Medicinal Chemistry, v. 58, p. 1964-1975, 2015.
Pertwee, R. G., The diverse CB1 and CB2 receptor pharmacology of three plant cannabinoids: Δ9-tetrahydrocannabinol, cannabidiol and A9-tetrahydrocannabivarin. British Journal of Pharmacology, v. 153, p. 199-215, 2008.
Ramachandran, G. N.; Ramakrishnan, C.; Sasisekharan, V., Stereochemistry of polypeptide chain configurations. Journal of Molecular Biology, v. 7, p. 95-99, 1963.
Santos, P. S. et al., Efeitos do ácido lipóico nas concentrações de glutamato e taurina no hipocampo de ratos após convulsões induzidas por pilocarpine. Arq. Neuro-Psiquiatr. [online], 2011, vol. 69, n.2b, pp. 360-364.
Munro et al., Nature 365:61-65, 1993.
Rinaldi-Carmona M. et al., J. Pharmacol. Exp. Ther. 278:871-878, 1996.
Gupta, A., Heimann, A. S., Gomes, I., Devi, L. A., Antibodies against G-protein coupled receptors: novel uses in screening and drug development. Comb. Chem High Throughput Screen. 2008, 11(6)463-467.
Langmead, C. J1., Watson, J., Reavill, C., Muscarinic acetylcholine receptors as CNS drug targets. Pharmacol Ther. 2008 February; 117(2):232-43. Epub 2007 Dec. 20.
Bomar, M. G. and Galande, A. K., Modulation of the cannabinoid receptors by hemopressin peptides. Life Sci. 2013 92(8-9): 520-524.
Dvoracsko, S., Tomboly, C., Berkecz, R., Keresztes, A., Investigation of receptor binding and functional characteristics of hemopressin(1-7). Neuropeptides 2016, 58:15-22.
Gomes, I., Grushko, J. S., Golebiewska, U., Hoogendoorn, S., Gupta, A., Heimann, A. S., Ferro, E. S., Scarlata, S., Fricker, L. D., and Devi, L. A., Novel endogenous peptide agonists of cannabinoid receptors. FASEB J. 2009 September; 23(9): 3020-3029.
Gelman, J. S., Sironi, J., Castro, L. M., Ferro, E. S., and Fricker, L. D., Hemopressins and other hemoglobin-derived peptides in mouse brain: Comparison between brain, blood, and heart peptidome and regulation in Cpefat/fat mice. J Neurochem. 2010 May; 113(4): 871-880.
Bauer, M., Chicca, A., Tamborrini, M., Eisen, D., Lerner, R., Lutz, B., Poetz, O., Pluschke, G., Gertsch, J., Identification and Quantification of a New Family of Peptide Endocannabinoids (Pepcans) Showing Negative Allosteric Modulation at CB 1 Receptors. The Journal of Biological Chemistry Vol. 287, No. 44, pp. 36944-36967, Oct. 26, 2012.
Reichwein, J. F. and Liskamp, R. M. J., Site-specific N-alkylation of peptides on the solid phase. Tetrahedron Letters, Volume 39, Issue 10, 5 Mar. 1998, Pages 1243-1246.
Szlavicz, E., Perera, P. S., Tomboly, C., Helyes, Z., Zador, F., Benyhe, S., Borsodi, A., Bojnik, E., Further Characterization of Hemopressin Peptide Fragments in the Opioid and Cannabinoid Systems. Anesth Analg. 2015 December; 121 (6): 1488-94. doi: 10.1213/ANE.0000000000000964. PubMed PMID: 26465932.
Special Periodic Reports, Amino Acids, Peptides and Proteins: Volume 42, Royal Society of Chemistry, 2013.

From the reviewed literature, no document was found anticipating or suggesting the teachings of the present invention, let alone their surprising technical effects, so that the solution proposed here, in the eyes of the inventors, has novelty and inventive step over the state of the art.

### BRIEF SUMMARY OF THE INVENTION

The present invention addresses a number of known problems of the prior art, for example, to provide: a peptide compound which is more stable and easier to handle than hemopressin; a synthetic intermediate useful for the preparation of compounds of pharmaceutical interest; the use of said compound to prepare a ligand of diagnostic interest of the cannabinoid and/or muscarinic system; the use of the compound to prepare an improved medicament relative to those containing hemopressin or cannabidiol; a pharmaceutical composition for modulating metabolic functions and/or for improved analgesic effect over compositions containing hemopressin; an immunomodulatory pharmaceutical composition; a neuromodulatory, neuroprotective pharmaceutical composition for the curative or prophylactic treatment of convulsions and/or multiple sclerosis; a therapeutic method; a molecular entity which provides these and/or other technical effects without the disadvantages arising from the use of hemopressin such as instability, formation of aggregates and/or low therapeutic effect and without the inconveniences arising from the use of cannabinoid substances, such as prostration and nasal bleeding, among others.

It is a further object of the invention to provide a peptidic compound having high stability at extremes of temperature and/or ease of handling, being particularly useful in pharmaceutical preparations and medicaments. The peptidic compound of the invention, among others, provides advantages in the administration, bioavailability and/or therapeutic action in an animal when compared to other peptides, such as hemopressin and known variants, usually of larger size (9 to 15 amino acids). The peptidic compound of the invention also provides oral administration to a mammalian animal and provides greater therapeutic effect than hemopressin, in addition to providing previously unknown uses and surprising in various aspects.

It is a further object of the invention to provide a compound substitute to cannabidiol and which provides advantages in the production, handling, use and safety and can replace it in all or almost all the applications already described therefor.

It is another object of the invention to provide a peptidic compound useful as a ligand of muscarinic and/or of cannabinoid system receptors. As such, the peptidic compound of the invention is useful for diagnostic applications. Another object of the invention is an in vitro process for diagnosing the presence, location and/or amount of cannabinoid and/or muscarinic receptors, as well as evaluating the binding of other compounds to such receptors.

The compound of the invention is also useful for modulating muscarinic and/or cannabinoid receptors, either by modulating the CB1 receptor, the CB2 receptor, both concomitantly, by modulating the binding or action of other substances interacting in the cannabinoid system, by the modulation of proteases or peptidases that lead to the generation or degradation of active peptides in the cannabinoid system, or combinations thereof.

Another object of the present invention is a synthetic intermediate in the preparation of compounds of pharmaceutical and/or diagnostic interest which comprise the peptidic compound of the invention and may include chemical modifications, substitutions, inclusion of other functional groups.

It is another object of the present invention to use the peptidic compound of the invention for the preparation of an improved pharmaceutical composition for modulating the metabolic functions of a mammal.

Another object of the present invention is the use of the peptidic compound of the invention for the preparation of an improved analgesic pharmaceutical composition.

It is another object of the present invention the use the peptidic compound of the invention for the preparation of a neuromodulator and/or neuroprotective medicament in a mammal.

It is another object of the present invention to use of the peptidic compound of the invention for the preparation of a medicament for the curative or prophylactic treatment of seizures in a mammal. In addition, administration of the peptidic compound of the invention to an animal provides important and surprising technical advantages, including superior anticonvulsant activity over cannabidiol and hemopressin, the use of which (hemopressin) as anticonvulsant being the subject matter of co-pending patent applications of the same inventors.

It is another object of the present invention the use of the peptidic compound of the invention for the preparation of a medicament for the curative or prophylactic treatment of multiple sclerosis in a mammal.

Other objects of the invention are therapeutic methods for: curative or prophylactic treatment of metabolic disorders, pain, seizures, multiple sclerosis, as well as for neuromodulation and/or neuroprotection.

Another object of the present invention is an improved pharmaceutical composition for modulating the metabolic functions of a mammal.

Another object of the present invention is an improved analgesic pharmaceutical composition.

Another object of the present invention is a neuromodulatory and/or neuroprotective pharmaceutical composition in a mammal.

Another object of the present invention is an improved pharmaceutical composition for the curative or prophylactic treatment of seizures in a mammal.

Another object of the present invention is a pharmaceutical composition for the curative or prophylactic treatment of multiple sclerosis in a mammal.

The compound of the invention is a peptidic compound of formula:

R₁—N-AA₁-K-AA₂-R₂

wherein:
AA₁ is an amino acid selected from the group consisting of F, W, L, I, V, P, G;
AA₂ is hydrogen or an amino acid selected from the group consisting of F, W, L, I, V, P, G;
R₁ is absent when R2 is the amino acid L, or is hydrogen or the amino acid V; and
R₂ is absent when AA₂ is hydrogen or is hydrogen, the amino acid L when R₁ is hydrogen, and/or modified forms thereof, cyclic-, amide- alkyl- alcoxy- halogen- hydroxyl-PEGylated forms thereof, forms modified with other functional groups, with amino acids or peptides, including non-natural amino acids, D-amino acids, its salts, and/or combinations thereof.

The compound of the invention is synthetic and distinct from known natural forms and is useful for preparing a product of pharmaceutical interest selected from a ligand for diagnostic use and a curative or prophylactic medicament for a mammal.

In one embodiment, AA₁ is F, W, or L.

In one embodiment, R₁ and R2 are both hydrogens.

In one embodiment, the compound of the invention is selected from the group consisting of: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, as well as modified or cyclic forms thereof, amidated, alkylated, alkoxylated, halogenated, hydroxylated, or PEGylated forms thereof, forms modified with other functional groups, as well as with an amino acid or peptide, including non-natural forms such as the d-amino acid forms, their salts; and/or combinations thereof.

In one embodiment, the peptidic compound of the invention is selected from the group comprising: the NFK tripeptide, the tetrapeptide of SEQ ID No. 1, the tetrapeptide of SEQ ID No. 7, as well as modified or cyclic forms thereof, amidated, alkylated, alkoxylated, halogenated, hydroxylated, PEGylated forms, forms modified with other functional groups, as well as with an amino acid or peptide, including the non-natural ones as the d-amino acid forms, their salts; and/or combinations thereof.

The pharmaceutical composition of the invention comprises the peptidic compound of the invention and a pharmaceutically acceptable vehicle, and may be in the form of a tablet, gel, oral liquid or syrup, capsule, suppository, injectable solution or inhalable or adhesive forms, optionally comprising other active ingredients.

These and other objects of the present patent application will be immediately appreciated by those skilled in the art and by companies having interests in this segment, and will be described in sufficient detail for its reproduction in the following description.

### BRIEF DESCRIPTION OF THE DRAWINGS

The following detailed description and the accompanying figures illustrate the main features and embodiments of the present invention, set forth in greater detail to provide further support to the person skilled in the art, and so that she/he may understand and reproduce the inventive concept of the invention in any of its embodiments. Such details or figures are not to be construed as limiting and serve only to illustrate some of the embodiments of the present invention.

FIG. 1 shows the results of comparative stability tests of the compound of the invention in the SEQ ID No. 1 embodiment vs. Hp (PVNFKFLSH). The data on the graph shows the results of the concentration measurement of each of these actives by HPLC, after freezing for 24 hours or separately after heating at 100° C. for 10 minutes (n=2). In the vertical axis the area of the HPLC peak is shown. In A) is shown the Hp (control); in B) Hp after freezing for 24 hours; in C) Hp heated to 100° C.; in D) SEQ ID No. 1 (control); in E) SEQ ID No. 1 after freezing for 24 hours; in F) SEQ ID No. 1 heated to 100° C. Statistical significance data are also shown, the asterisks indicating: (*) P<0.05 vs Control (***) P<0.005 vs Control; (****) P<0.0001 vs Control.

FIG. 2 shows the test results of the compound of the invention in the SEQ ID No. 1 embodiment compared to the hemopressin test results, both in the pilocarpine model. The % of time in relation to the control for the occurrence of the first salivation is presented with the administration of the following treatment doses: In A) the control (saline); in B) hemopressin (Hp or PVNFKFLSH, 0.551334 μmol/kg); in C) hemopressin (0.91889 μmol/kg); in D) the compound of the invention in the SEQ ID No. 1 embodiment (0.540882 μmol/kg); in E) SEQ ID No. 1 (0.901469 μmol/kg); in F) the compound PEP-19 (DIIADDEPLT, 0.908117 μmol/kg). The asterisks indicate the statistical significance: (*) P<0.05 vs Control; (**) P<0.01 vs Control; the + sign indicates P<0.05 vs Hp 0.91889 μmol/kg.

FIG. 3 shows the test results of the compound of the invention in the SEQ ID No. 1 embodiment as an anticonvulsant compared to the results of hemopressin tests as an anticonvulsant, both in the pilocarpine model. The percentage of time (relative to the control) for the first seizure is presented with the administration of the following treatment doses: in A) the control (saline); in B) hemopressin (Hp or PVNFKFLSH, 0.551334 μmol/kg); in C) hemopressin (0.91889 μmol/kg); in D) the compound of the invention in the SEQ ID No. 1 embodiment (0.540882 μmol/kg); in E) SEQ ID No. 1 (0.901469 μmol/kg); in F) the compound PEP-19 (DIIADDEPLT, 0.908117 μmol/kg). The asterisks indicate the statistical significance: (*) P<0.05 vs Control; (**) P<0.01 vs Control; the + sign indicates P<0.05 vs Hp 0.91889 μmol/kg.

FIG. 4 shows the test results of the compound of the invention in the SEQ ID No. 1 embodiment in the pilocarpine model, being indicated the time for occurring first salivation with the administration of: A) control; B) cannabidiol (30 mg/kg); or C) SEQ ID No. 1 (500 μg/kg). The vertical axis shows the time in minutes. The data between control and other test compounds does not present statistical significance among themselves under the conditions tested.

FIG. 5 shows the test results of the compound of the invention in the SEQ ID No. 1 embodiment as an anticonvulsant in the pilocarpine model, being indicated the time for occurring the first convulsion with the administration of: A); control B) cannabidiol (30 mg/kg); or C) SEQ ID No. 1 (50 μg/g). The vertical axis shows the time in minutes. The asterisks indicate the statistical significance: (**) P<0.02 vs Control; (***) P<0.002 vs Control.

FIG. 6 shows the results of neuroprotection/survival tests with the compound of the invention in the SEQ ID No. 1 embodiment in the pilocarpine model, the survival/death profile of the animals being indicated by the administration of SEQ ID No. 1. In the vertical axis the number of survivors is shown. The horizontal axis shows the time in minutes. It is shown: in A) the survival profile of the animals administered with the control (saline only; in B) the survival profile of the animals administered with cannabidiol 30 mg/kg is shown; in C) the survival profile of animals given SEQ ID No. 1 500 μg/kg; in D) all profiles are shown in a single graph. It is interesting to note that in the group treated with the neuromodulator SEQ ID No. 1 500 μg/kg only two animals died. The remaining animals in the SEQ ID No. 1 group, for a total of 3, remained alive for more than a week, while practically all the others died in less than 30 minutes. Consequently, the mean survival time for this group is significantly different and much higher than the others. In addition, when comparing the SEQ ID No. 1 group (500 μg/kg) with the group administered with cannabidiol (30 mg/kg), the survival rate is three times higher in the first group, that is, in the SEQ ID No. 1 500 μg/kg group, even using a 60-fold lower relative concentration of this compound.

FIG. 7 shows the results of neuroprotection tests with different embodiments of the invention compound (SEQ ID No. 1, SEQ ID No. 7 and NFK, all in 600 μg/kg), previously administered to groups of animals (n=5 in each group) and subsequently administration of pilocarpine, showing the survival/death profiles of the animals after 24 hours in the model tested. The vertical axis shows the number of dead animals. The control is the administration of saline prior to the administration of pilocarpine.

FIG. 8 shows the test results of the compounds of the invention SEQ ID No. 1, SEQ ID No. 7, NFK, FKF, FKL, as well as the dipeptides NF, FK, KF and KL in the pilocarpine model, indicating the time (min) for the occurrence of the first salivation after the administration of control or the test compounds. * P<0.05 vs Control. The results reflect the mean results of seven animals in the control group and six animals in the group treated with NFK.

FIG. 9 shows the test results of the compounds of the invention SEQ ID No. 1, SEQ ID No. 7, NFK, FKF, FKL, as well as the dipeptides NF, FK, KF and KL in the pilocarpine model, indicating the time (min) for the occurrence of the first signal after administration of control or test compounds.

FIG. 10 shows the test results of the compounds of the invention SEQ ID No. 1, SEQ ID No. 7, NFK, FKF, FKL, as well as the dipeptides NF, FK, KF and KL in the pilocarpine model, indicating the time for the occurrence of seizures after administration of control or test compounds. * P<0.05 vs control; ** P<0.001 vs control.

FIG. 11 shows the test results of the compounds of the invention SEQ ID No. 1, SEQ ID No. 7, NFK, FKF, FKL, as well as the dipeptides NF, FK, KF and KL in the pilocarpine model, indicating the time (min) for the occurrence of death after administration of control or test compounds. * P<0.05 vs control.

FIG. 12 shows results of the binding assays of various compounds to the CB1 receptor in anti-CB1 antibodies sensitive to activation/conformational changes of receptor. Treatment with 1 μM. The values in % of binding to the CB1 receptor in relation to control are indicated. * P<0.05 vs control.

FIG. 13 presents an overview of the three-dimensional structure of a GPCR, in this case, the cannabinoid receptor of subtype 1. Reference is made to the seven transmembrane helices (I-VII), the intracellular loops (ICL1 and ICL2) and the extracellular loops (ECL2 and ECL3).

FIG. 14 shows the overlap of AM6538 structure at the crystallographic structure (PDB 5TGZ), and the result obtained after validation by the Goldscore function (when the figure appears colored, the crystal structure is purple and the result of the Goldscore function in blue/cyan, although for the purposes of this application such colors are irrelevant).

FIG. 15 shows the major interactions observed for AM6538 at the CB1 receptor binding site.

FIG. 16 shows the main interactions observed for rimonabant at the CB1 receptor binding site.

FIG. 17 shows the major interactions observed for cannabidiol at the CB1 receptor binding site.

FIG. 18 shows the major interactions observed for the peptidic compound of the invention in the SEQ ID No. 1 embodiment at the CB1 receptor binding site.

FIG. 19 shows the results of the process of obtaining CB2 receptor structure. In A) the three-dimensional structure of the obtained CB2 receptor is shown; in B) the Ramachandran Graph for the obtained human CB2 model is shown.

FIG. 20 shows the major interactions between the CB2 receptor and the AM6538 ligand.

FIG. 21 shows the major interactions between the CB2 receptor and rimonabant.

FIG. 22 shows the major interactions between the CB2 receptor and cannabidiol.

FIG. 23 shows the major interactions between the CB2 receptor and the compound of the invention in the SEQ ID No. 1 embodiment.

FIG. 24 shows the pain threshold results, represented as force in grams, applied to the back of the right paw of rats. When animals react by kicking, the force in grams required to induce this response is the pain threshold. The vertical axis shows the pressure applied at the leg. Antinoniceptive activity was expressed as the increase in pain threshold compared to control animals. The compounds of the invention were administered orally to animals at doses of 0.5 to 0.25 mg/kg and saline was used as a control. * P<0.05 vs control.

FIG. 25 shows the test results of the compound of the invention in the SEQ ID No. 1 embodiment for the control of multiple sclerosis. The vertical axis shows the clinical score. The horizontal axis shows the number of days after immunization. A) shows the Clinical Score curve of the Wild Type (WT) mouse, that is, normal, submitted to EAE induction with MOG; B) shows the Clinical Score curve of the knock out mouse of the endopeptidase gene 24.15, that is, transgenic and prone to Multiple Sclerosis symptoms, in the EAE model, submitted to MOG induction; C) shows the Clinical Score curve of the knock out mouse of the endopeptidase gene 24.15, that is, transgenic and prone to Multiple Sclerosis symptoms, in the EAE model, submitted to MOG induction and the neuroprotection obtained with SEQ ID No. 1 * P<0.05 24.15 KO+SEQ ID No. 1 vs WT; +P<0.05 24.15 KO vs WT.

### DETAILED DESCRIPTION OF PREFERRED EMBODIMENTS

The inventive concept underlying the several objects of the invention is a peptidic compound of formula:

R₁—N-AA₁-K-AA₂-R₂

wherein:
AA₁ is an amino acid selected from the group consisting of F, W, L, I, V, P, G;
AA₂ is hydrogen or an amino acid selected from the group consisting of F, W, L, I, V, P, G;
R₁ is absent when R₂ is the amino acid L, or is hydrogen or the amino acid V; and
R₂ is absent when AA₂ is hydrogen or is hydrogen, the amino acid L when R₁ is hydrogen,
and/or modified forms thereof, cyclic-, amide- alkyl- alcoxy- halogen- hydroxyl-PEGylated forms thereof, forms modified with other functional groups, with amino acids or peptides, including non-natural amino acids, D-amino acids, its salts, and/or combinations thereof.

The compound of the invention is synthetic and distinct from known natural forms and is useful for preparing a product of pharmaceutical interest selected from a ligand for diagnostic use and a curative or prophylactic medicament for a mammal.

In one embodiment, AA₁ is F, W, or L.

In one embodiment, R₁ and R₂ are both hydrogen.

In one embodiment, the compound of the invention is selected from the group consisting of: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, as well as modified or cyclic forms thereof, amidated, alkylated, alkoxylated, halogenated, hydroxylated, or PEGylated forms thereof, forms modified with other functional groups, as well as with an amino acid or peptide, including non-natural forms such as the d-amino acid forms, their salts; and/or combinations thereof.

In one embodiment, the peptidic compound of the invention is selected from the group comprising: the NFK tripeptide, the tetrapeptide of SEQ ID No. 1, the tetrapeptide of SEQ ID No. 7, as well as modified or cyclic forms thereof, amidated, alkylated, alkoxylated, halogenated, hydroxylated, PEGylated forms, forms modified with other functional groups, as well as with an amino acid or peptide, including the non-natural ones as the d-amino acid forms, their salts; and/or combinations thereof.

Said peptidic compound has surprisingly high stability at extremes of temperature and ease of handling, being particularly useful in pharmaceutical preparations and medicaments. The peptidic compound of the invention, among others, provides advantages in the administration, bioavailability and/or therapeutic action in an animal when compared to other peptides, such as, for example, hemopressin and known variants, usually of larger size (9 to 23 amino acids). The peptidic compound of the invention also provides for oral administration to a mammalian animal and provides a greater therapeutic effect than those known for hemopressin and may advantageously replace hemopressin in all or almost all applications already described therefor.

The compound of the invention is also a synthetic intermediate in the preparation of compounds of pharmaceutical interest which comprise the peptidic compound of the invention and include chemical modifications, substitutions, inclusion of other functional groups.

The compound of the invention is an advantageous substitute for cannabidiol and provides advantages in the production, handling, use and safety and is useful for replacing cannabidiol in all or almost all applications already described therefor.

The compound of the invention is a ligand of muscarinic and/or the cannabinoid system receptors, being useful for diagnostic applications or for modulating muscarinic and/or cannabinoid receptors, either by modulation of the CB1 receptor, CB2 receptor, both concomitantly, by modulating the binding or action of other substances interacting in the cannabinoid system, by modulating proteases or peptidases leading to the generation or degradation of active peptides in the cannabinoid system, or combinations thereof. The test results presented in the present patent application show that the compound of the invention interacts with and/or modulates the activity of cannabinoid and/or muscarinic receptors.

The compound of the invention is useful in an improved pharmaceutical composition for modulating the metabolic functions of a mammal.

The compound of the invention is useful in an improved analgesic pharmaceutical composition.

The compound of the invention is useful in a pharmaceutical composition for the curative or prophylactic treatment of seizures in a mammal. Administration of the peptidic compound of the invention to an animal provides important and surprising technical advantages, including superior anticonvulsant activity over cannabidiol and hemopressin, the use of the latter as an anticonvulsant being the subject of co-pending patent application PCT/BR2017/050313, of the same inventors.

The compound of the invention is useful in a neuromodulatory and/or neuroprotective pharmaceutical composition in a mammal. The test results presented in the present patent application show that the composition of the invention modulates action of neurons and is also neuroprotective, anticonvulsive and is useful in the treatment of multiple sclerosis.

For purposes of the present invention the following definitions are used.

Product of Pharmaceutical Interest

In the context of the present application, “compound of pharmaceutical interest” means any molecular entity comprising the compound described as inventive concept common to the present application, including also molecular entities obtained by chemical modification/derivatization of the same, or with the inclusion of other functional groups, linear or branched side chains, alteration of hydrophilicity or hydrophobicity, among others, provided that they comprise as nucleus the entity R1-N-AA1-K-AA2-R2 as defined above, except for the natural and already known entities.

Pharmaceutical Composition

In the context of the present patent application, “pharmaceutical composition” is to be understood as any and all compositions containing an active principle, for prophylactic, palliative and/or curative purposes, acting in a manner to maintain and/or restore the homeostasis, and may be administered orally, topically, parenterally, enterally and/or intrathecally.

Pharmaceutically Acceptable Formulation

In the context of the present application a “pharmaceutically acceptable formulation” is meant as a formulation containing pharmaceutically acceptable excipients and carriers well known to those skilled in the art, such as the development of convenient doses and treatments for use in particular compositions which can be described in a number of treatment regimens, including oral, parenteral, intravenous, intranasal, intravitreal and intramuscular, intracerebral, intracerebroventricular and intraocular treatment and administration and/or formulation.

Modified Peptide

In the context of the present application, “modified peptide” is to be understood as a non-naturally occurring, artificially modified or synthesized peptide, including halides, cyclized, amidated, alkylated, alkoxylated, hydroxylated, PEGylated forms, forms with other functional groups on any amino acid, or salt forms thereof, as well as on an amino acid or peptide, including the non-natural, such as d-amino acid forms. The peptidic compound may be pegylated using techniques known to those skilled in the art, such as, for example, pegylation with reagents containing the succinimidyl group, which preferentially react with primary amines present in the N-terminal region of the peptide. The peptidic compound of the invention may be alkylated at any amino acid using techniques known to those skilled in the art, including, for example, the Mitsunobu reaction described in Reichwein and Liskamp (Reichwein J F and Liskamp R M J, Site-specific N-alkylation of peptides on the solid phase, Tetrahedron Letters, Volume 39, Issue 10, 5 Mar. 1998, Pages 1243-1248). Said article describes the introduction of any alkyl group into a specific amide function of a peptide. The peptidic compound of the invention may be alkoxylated, substituted with halogens, hydroxy or other functional groups on any amino acid using techniques known to those skilled in the art, including, for example, those described in the book Special Periodic Reports, Amino Acids, Peptides and Proteins: Volume 42, Royal Society of Chemistry, 2013. The peptidic compound of the invention may be modified with other molecular species useful in diagnostic and/or therapeutic applications, such as Biotin, using techniques known to those skilled in the art.

Cyclic or Circular Peptide

In the context of the present application, “cyclic, cyclized or circular peptide” is to be understood as a peptide which has a covalent bond between the two ends of a linear peptide molecule by any method known in the art, particularly by the activity of enzymes. The cyclic peptide can be used instead of the linear peptide because it is more difficult to be degraded, since its ends or zones of attack by hydrolyzing enzymes are not as exposed as in a linear peptide.

Agonist

In the context of the present application, “agonist” is to be understood as a drug, drug, hormone, neurotransmitter or other signaling molecule which forms a complex with a receptor site, thereby triggering an active response of a cell.

Inverse Agonist/Antagonist

In the context of the present application, “inverse agonist or antagonist” shall be understood as agent(s) (for example, drugs, drugs, hormones or enzymes) which bind(s) to agonist receptors and produce(s) pharmacological effects opposite to those of the agonists, such that the action of one partially or totally inhibits the effect of the other. Particularly, a compound is an inverse agonist when it acts in the presence of an agonist, but reducing its activity; an antagonist is a compound that will totally block the activity of the agonist.

Equivalent Dose in Humans

In the present invention, the concept of “equivalent dose in humans” is the dose at which, in humans, the same magnitude of effects is expected in animals at a given dose, as set forth in “Guidance for Industry Estimating the Maximum Safe Starting Dose in Initial Clinical Trials for Therapeutics in Adult Healthy Volunteers” published by the US Department of Health and Human Services Food and Drug Administration Center for Drug Evaluation and Research (CDER), July 2005 Pharmacology and Toxicology. In said guide, the conversion of the observed dose in animals (mg/kg) to Equivalent Dose in Humans (mg/kg) entails dividing the result obtained in rats by 6.2 and the result obtained in mice by 12.3. These values are applicable to a human of 60 kg standard weight. For other species or for weights outside the standard weight ranges, the Dose Equivalent in Humans (DEH) can be calculated by the formula: DEH=dose in animal in mg/kg×(animal weight in kg/human weight in kg)^{0.33}. This Guidance considers a safety range of 10 times the concentration limits tested to be adequate.

Ligand of CB Receptor

In the context of the present patent application, a “ligand of CB receptor” is to be understood as a compound or molecule that interacts with the CB system and/or with CB1 or CB2 receptors.

Modulating the CB Receptor Function or the Cannabinoid System

In the context of the present patent application, it is meant to “modulate the CB receptor function” as an interaction that results in the alteration of the biochemical activity of the CB receptor, particularly CB1 or CB2. It is understood that the change is positive when an antagonist or inverse agonist effect occurs at CB receptors and that the change is negative when an agonist effect occurs at CB receptors. The results of the tests presented in the present patent application indicate that the compound of the invention interacts with and/or modulates the CB1 receptor and/or the CB2 receptor, probably as an allosteric modulator of CB1 and/or CB2 receptor. Thus the compound of the invention is useful for modulating the cannabinoid system, either by modulating the CB1 receptor, or the CB2 receptor, of both concomitantly, by modulating the binding or action of other substances interacting in the cannabinoid system, by modulating proteases or peptidases which lead to the generation or degradation of active peptides in the cannabinoid system, or by improving the natural system by the protection of natural molecules with the compounds of the invention, that is, the compound of the invention could also lead the natural system to a protective action independently of the binding to said receptors, or combinations of said effects.

Modulating the Function of Muscarinic Receptors

In the context of the present application, the term “modulating the function of muscarinic receptors” should be understood as an interaction which leads to alterations in muscarinic acetylcholine receptors (mAChRs), which play an important role in cognitive functions, such as learning and memory, control of dopamine release, modulation of locomotor activity, its modulation being also useful in the control of Alzheimer's disease and/or control of dependency or addiction to abuse drugs. It is understood that the change is positive when an antagonist or inverse agonist effect occurs at muscarinic receptors and that the change is negative when an agonist effect occurs at muscarinic receptors. The tests presented in the present patent application suggest that the compound of the invention interacts with and/or modulates muscarinic receptors.

Modulation of Metabolic Functions

In the context of the present application, this term is understood to include modulation of: energy and/or lipid metabolism; arterial hypertension, regulation of intestinal motility; immune system; balance of the calcium cycle, conditions associated with the thyroid gland, peripheral organs and tissues, including reproductive organs, adipose tissue, liver, muscles and gastrointestinal tract, being useful in the treatment of obesity, diabetes, diseases or immune/inflammatory disorders, osteopenia, osteoporosis, cancer. In this context, the peptidic compounds of the invention are also useful in pharmaceutical compositions for the treatment of metabolic disorders comprising preventing overweight; regulation of appetite; induction of satiety; prevention of weight gain after successful weight loss; increased energy consumption; aesthetic weight reduction; or bulimia.

Neuromodulator

In the context of the present application the term “neuromodulator” or “neuromodulation” is intended to modulate neuronal/neurological function, including modulation of brain, cortex, hippocampus, amygdala, pituitary, hypothalamic activity; adrenal gland. Neuromodulation includes the beneficial modulation of neuroprotection against agents or conditions leading to pathophysiological processes. Neuroprotective agents or compounds are preferably used prior to (or during) the prodromal stage of disease, which often begins many years before the onset of symptoms. In the present invention, a neuromodulator is potentially useful in the curative or prophylactic treatment of a variety of neurological conditions or diseases, including essential tremor, migraine, pain, neuropathic pain, multiple sclerosis, amyotrophic lateral sclerosis, psychiatric disorders such as anxiety, schizophrenia or bipolar disorder, congenital disorders such as dementia, Alzheimer's, Parkinson's, autism and also potentially useful in modifying the pathophysiological processes involved in the occurrence of seizures and/or epilepsy, as well as in other clinical conditions related to disorders of neuronal excitability or neuronal lesions due to ischemia, hypoxia or other harmful conditions.

Although in this patent application it is demonstrated that the compound of the invention binds and/or modulates cannabinoid receptors, the surprising pharmaceutical action of the invention may be linked to action on CB1 and/or CB2 and/or muscarinic receptors or possibly be linked to modulation of uptake of adenosine, GGPR55, PPARγ receptors, intracellular calcium level, modulation of opioid receptors that form heterodimers with cannabinoid receptors, or combinations thereof. Thus, any therapeutic indication related to these targets may benefit from the present invention.

The present invention is also defined by the following clauses.

Peptidic compound described above.

Use of the peptidic compound described above for the preparation of a product of pharmaceutical interest selected from a ligand for diagnostic use and a curative or prophylactic medicament in a mammal.

Use as described above wherein AA1 and/or AA2 is F, W or L.

The use as described above wherein said peptidic compound is selected from the group consisting of: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, as well as modified, cyclic forms, amidated, alkylated, alkoxylated, halogenated, hydroxylated, PEGylated forms, forms modified with other functional groups, as well as with an amino acid or peptide, including the non-natural such as d-amino acid forms, their salts; and/or combinations thereof.

The use as described above wherein said peptidic compound is selected from the group consisting of: the NFK tripeptide, the tetrapeptide of SEQ ID No. 1, the tetrapeptide of SEQ ID No. 7, as well as modified, cyclic forms thereof, amidated, alkylated, alkoxylated, halogenated, hydroxylated, PEGylated forms, forms modified with other functional groups, as well as with an amino acid or peptide, including the non-natural as the d-amino acid forms, their salts; and/or combinations thereof.

Synthesis intermediate in the preparation of compounds of pharmaceutical interest comprising the compound described above.

Use of the above-described compound as a synthetic intermediate in the preparation of other compounds of pharmaceutical and/or diagnostic interest.

The compound described above, modified.

Use of the compound described above for preparation of a cannabinoid receptor ligand and/or muscarinic receptors.

Use of the compound described above for the preparation of a binder of diagnostic interest in a mammal.

Use of the compound described above for the preparation of a pharmaceutical composition modulating metabolic functions.

Use of the compound described above for the preparation of an analgesic pharmaceutical composition.

Use of the compound described above for the preparation of an immunomodulatory pharmaceutical composition.

Use of the compound described above for the preparation of a neuromodulator and/or neuroprotective medicament in a mammal.

Use of the compound described above for the preparation of a curative or prophylactic anticonvulsant drug in a mammal.

Use of the compound described above for the preparation of a medicament for the curative or prophylactic treatment of Multiple Sclerosis in a mammal.

A pharmaceutical composition modulating metabolic functions in a mammal comprising a pharmaceutically acceptable carrier; and, as active principle, the compound described above.

Analgesic pharmaceutical composition in a mammal comprising a pharmaceutically acceptable carrier; and, as active principle, the compound described above.

A neuromodulatory and/or neuroprotective pharmaceutical composition comprising a pharmaceutically acceptable carrier; and, as active principle, the compound described above.

A pharmaceutical composition for the curative or prophylactic treatment of seizures in a mammal comprising a pharmaceutically acceptable carrier; and, as active principle, the compound described above.

Pharmaceutical composition for the curative or prophylactic treatment of Multiple Sclerosis in a mammal comprising a pharmaceutically acceptable carrier; and, as active principle, the compound described above.

A pharmaceutical composition comprising the NFK tripeptide, the tetrapeptide SEQ ID No. 1, the tetrapeptide of SEQ ID No. 7, or combinations thereof.

Pharmaceutical composition as described above in the form of a tablet, tablet, gel, oral liquid or syrup, capsule, suppository, solution for injection, inhalable form or in adhesive.

A therapeutic modulator of metabolic functions of an animal, comprising administering the peptide described above. Particularly for this application, the peptide of the invention is preferably modified so that its molecular weight is greater, minimizing or preventing the passage thereof through the blood-brain barrier.

A therapeutic method for the curative or prophylactic treatment of pain comprising administering to an animal the compound described above.

Therapeutic method for curative or prophylactic neuromodulation and/or neuroprotection comprising administering to an animal the compound described above,

Therapeutic method for the curative or prophylactic treatment of seizures comprising administering, to an animal, the compound described above.

Therapeutic method for the curative or prophylactic treatment of Multiple Sclerosis comprising administering to an animal the compound described above.

The results of in vitro and in silico tests have shown that the compound of the invention provides advantages in the preparation of pharmaceutical compositions, in their stability, and in therapeutic effects.

The compound of the invention has proved to be much more stable than hemopressin, which in addition causes technical problems of fiber formation or fibrils, as well as its known variants.

The results of in vivo tests in mammals showed excellent therapeutic effect at low dosages, with no evidence of significant side effects

The peptidic compound of the invention has also been shown to be an appropriate synthetic intermediate for the preparation of other compounds useful in diagnostic and/or therapeutic applications. Tests conducted in the present invention show that the compound of the invention is suitably modified with biotin known to those skilled in the art as useful in diagnostic preparations or kits. The peptidic compound of the invention, when modified/protected by biotin, has also been shown to bind to the CB1 receptor in tests with anti-CB1 antibodies sensitive to the activation/conformational changes of the receptor. Another object of the invention is an in vitro method of diagnosing the presence, amount and/or localization of cannabinoid, opioid and/or muscarinic receptors, as well as evaluating the binding of other compounds to these receptors.

The neuromodulator/neuroprotective effect resulting from the administration of the compound of the invention in mammals is evidenced by the decrease and/or absence of symptoms, brain damage and death associated with the administration of substances known to be harmful, such as pilocarpine. Tests with the EAE/MOG model, the most commonly used model for replicating multiple sclerosis, also show that administration of the peptide compound of the invention to an animal substantially diminishes the clinical symptoms of multiple sclerosis, a disease essentially related to neuronal damage.

In one embodiment, the invention provides the use of said compound for the preparation of a neuromodulatory, neuroprotective medicament and/or for the curative or prophylactic treatment of seizures in a mammal. Administration, to an animal, of the compound of the invention provides neuromodulatory, neuroprotective, anticonvulsive and/or symptomatic multiple sclerosis inhibiting activity; makes oral administration feasible; does not have or entails the drawbacks arising from the production, storage, transport and use of cannabinoid substances, in addition to providing additional advantages in the preparation of therapeutic products for mammals in their administration and/or effects. In addition, administration of the compound of the invention to an animal provides important and surprising technical advantages, including superior anticonvulsant activity with respect to hemopressin, the use of which as an anticonvulsant is the subject of the co-pending application of the same inventors.

The present invention describes the use of a compound for the preparation of pharmaceutical compositions useful for a wide variety of medical conditions, including those related to the central nervous system. Surprisingly, although the active compound of the invention is peptidic or predominantly peptidic, oral administration has provided brain effects in animals.

In one embodiment, in vivo tests with the composition of the invention have shown surprising results as to its neuroprotective activity. The composition of the invention, when administered previously to animals, provided surprising, potent and long protection against damage resulting from the subsequent administration of substances known to be harmful to neurons. Accordingly, the neuroprotection provided by the compound of the invention is particularly useful as a therapeutic alternative for various medical conditions, including those related to disorders of neuronal excitability, such as seizures.

In various embodiments, in vivo tests with the compound of the invention demonstrated surprising results regarding its anticonvulsant activity. The compound of the invention, when used as an anticonvulsant, additionally provides the advantage of being a good candidate to substitute for the cannabinoid compounds known to act as anticonvulsants, such as cannabidiol. Cannabidiol, despite its proven effects as an anticonvulsant, has been facing regulatory problems due to its origin, the Cannabis sativa plant. The present invention provides an additional therapeutic approach for patients suffering from seizures and having difficulty in obtaining drugs, being based on a peptide, ie, does not use derivatives of Cannabis sativa. In addition, the results showed that the compound of the invention, when used as an anticonvulsant, provides other surprising technical advantages in use, including greater therapeutic effect, oral use, lower dosage, less occurrence of side effects such as prostration and nasal bleeding, among others technical advantages.

The molecular mechanism of how cannabidiol (CBD) ceases seizures is not yet fully understood. It is known that CBD: inhibits adenosine reuptake; is a 5-HT 1A agonist; is an antagonist of GPR55 (CB3); is a PPARγ receptor agonist; increases intracellular Ca2+, in addition to interacting with CB1 and CB2. Hemopressin, on the other hand, has a predominant action via pERK1/2 and AKT. It is therefore surprising and at a difficult time to explain why both the peptidic compound of the invention and the CBD have anticonvulsive action.

In the experimental convulsion model tests, administration of the pharmaceutical composition of the invention to mammals resulted in excellent therapeutic effect at low dosages when compared to dosages of a reference compound, cannabidiol. In addition, test results have shown that the composition of the invention provides surprising technical advantages in use, including greater therapeutic effect, viability of oral use, lack of use of carrier oil (which in many cases causes side effects), lower dosage and lower occurrence of side effects such as prostration and nasal bleeding, among others.

In vivo comparative tests demonstrate that the compound of the invention has superior activity and/or requires less dosage than hemopressin, the use of which as an anticonvulsant is the subject of the co-pending patent application of the same inventors.

The pharmaceutical composition of the invention is also useful for the treatment of diseases associated with modulation of the activity of the cannabinoid system, cannabinoid (CB) and/or muscarinic receptors—with several technical advantages and without known undesirable effects of the available congeners in the state of the art.

Additionally, as will be demonstrated in the following examples, the use of the compound of the invention in the preparation of a neuromodulatory, neuroprotective and/or anticonvulsant drug provides for the delivery of a medicament orally administrable to a mammal. Test results revealed significant brain action, suggesting that administration of the compound of the invention allows the active element to cross the blood brain barrier Thus, the results show/support the use of the compound of the invention regardless of whether the compound of the invention is the active which acts directly on the target, i.e. does not degrade during oral ingestion, or the compound is a precursor which, upon modification chemistry, acts on the target—in this case, being characterized as a pro-drug. This feature of providing important effects even by oral administration is particularly desirable, since the natural enzymes of a mammal in general degrade peptides and proteins while in the digestive tract and rarely a drug with a peptidic active is shown to be viable. However, surprisingly, the compound of the invention—even when administered orally—provides strong therapeutic action, in this case even more surprisingly, acting in the brain.

Thus, regardless of the mechanism of action, which is not the subject of the present application, the oral administration of the pharmaceutical composition of the invention has provided important neuromodulatory, neuroprotective, anticonvulsive, pain-modulating and symptomatic multiple sclerosis and even avoiding deaths, clearly shows the surprising magnitude and relevance of solved technical problems.

The pharmaceutical composition of the invention comprises the compound described above and also a pharmaceutically acceptable carrier, optionally also comprising other pharmaceutically acceptable actives and/or salts thereof. The pharmaceutical composition of the invention may be administered in the form of a tablet, gel, capsule, oral liquid or syrup, a suppository, an injectable solution or other suitable forms of administration for pharmaceutical and medical purposes.

The following examples are only intended to exemplify some of the various ways of practicing the invention, however, without limiting the scope thereof.

Comparative tests showed that the compound of the invention showed superior in vitro stability at temperature extremes when compared to hemopressin.

In some examples, the neuromodulatory/neuroprotective/anticonvulsive effects of the composition of the invention have been evaluated in vivo by oral administration to animals. The pharmaceutical composition of the invention was administered to mammals (Mus musculus or mouse) with an oral dose of treatment with different embodiments of the compound of the invention, compared to other compounds or to saline control. In these experiments, the test compounds were administered orally 10 minutes prior to (intraperitoneal) administration of pilocarpine. Pilocarpine hydrochloride (320 mg/kg, Merck), dissolved in 0.9% sterile saline, was given intraperitoneally for induction of SE (status epileticus) (Turski et al., 1983). In the Turski model, the neurotoxic effects begin about 15-25 minutes after the injection of Pilo, with the occurrence of motor and limbic seizures, the animals evolving to a state of continuous (clonic) seizures that characterize SE Sanabria and Cavalheiro, 2000).

Comparative tests also showed that the compound of the invention showed superior in vivo therapeutic activity when compared to hemopressin, the use of which as an anticonvulsant is the subject of co-pending patent application PCT/BR/2017050313, the same inventors.

The compound of the invention, being a ligand/modulator of an important GPCR (cannabinoid receptor), is useful in modulating GPCR receptor activity under pathological conditions, as well as modulating the target GPCR and also as a carrier of other targeted therapeutic molecular entities to cells expressing GPCRs that dimerize with cannabinoid receptors. The compound of the invention is also useful in therapies combined with antibodies, especially monoclonal antibodies, selective for the activated/modulated conformation of the receptor. Such therapies provide the advantage of conferring high specificity, potentially reducing also the dose of administration.

### EXAMPLES

### Example 1. Stability Tests Under Extreme Conditions—Superiority of the Compound of the Invention in Relation to Hemopressin

In this embodiment, the stability of the compound of the invention in the SEQ ID No. 1 embodiment was compared to that of hemopressin (Hp, PVNFKFLSH) under extreme conditions. Hp is known to have the problem of fibril formation, as well as variants thereof which have a greater number of amino acids. Samples of SEQ ID No. 1 and Hp were subjected to two separate tests, that of stability by freezing for 24 hours and heating at 100° C. for 10 minutes.

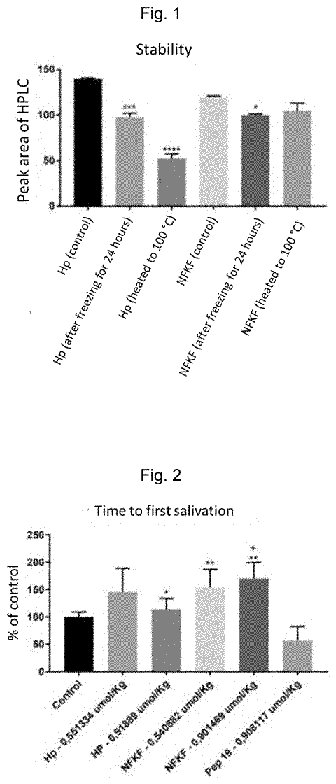

The results of FIG. 1 show that, by freezing for 24 h, a significant part of Hp is lost or degraded (from 140 to 97, i.e., approximately 31%), as evidenced by measurements by HPLC (analytical column of 2.1 mm, with gradient running of 10-60% B. Solvent A is water/0.1 TFA and solvent B is acetonitrile/0.075% TFA). The compound of the invention in the SEQ ID No. 1 embodiment, on the other hand, remained much more stable and suffered substantially less degradation (from 120 to 100, i.e., 16.7%). The results of FIG. 1 also show that upon heating at 100° C. for 10 minutes, even more significant part of the Hp is lost or degraded (from 140 to 50, i.e., approximately 64.3%), as evidenced by measurements by HPLC. On the other hand, the compound of the invention in the SEQ ID No. 1 embodiment remained stable and did not suffer statistically significant degradation.

Together, these data show that the compound of the invention provides much greater stability and lower degradation under extreme temperature conditions, which favors it in the manipulation, galenics, pharmaceutical preparations and stability of the pharmaceutical both in the post-acquisition phase of the active principle, and in the industrial production of medicines and, not least, in the transport logistics chain. The data suggest that the shelf life of the pharmaceutical product containing the compound of the invention should be greater than the congeners containing Hp or other actives with instability problems.

### Example 2, Use of Compound R1-N-AA1-K-AA2-R2 for the Preparation of Pharmaceutical Composition

In this embodiment, the compound R₁—N-AA₁-K-AA₂-R₂ is the tetrapeptide SEQ ID No. 1, which has been synthesized by chemical synthesis. Said peptide has been used in the preparation of an oral liquid pharmaceutical composition comprising between 2.7×10⁻⁴ Molar of said peptide and a pharmaceutically acceptable carrier. In this embodiment, said carrier is saline, the pharmaceutical composition being a solution for oral use. Said composition was used for in vivo oral administration to mammals according to Examples 3-8 below.

In other embodiments, the pharmaceutical composition is in the form of a tablet, gel, oral liquid or syrup, capsule, suppository, injectable solution or inhalable or adhesive forms, optionally comprising other active principles.

### Example 3. Comparative Pharmaceutical Composition Comprising the Compound of SEQ ID No. 1 with the Pharmaceutical Composition Comprising Hp—Results of In Vivo Tests

In this example, the effects of the composition of the invention with respect to the pharmaceutical composition containing Hemopressin (Hp or PVNFKFLSH), the use of which as anticonvulsant is co-pending patent application PCT/BR2017/050313, of the same inventors, were compared.

FIG. 2 shows the test results of the compound of the invention SEQ ID No. 1 compared to the hemopressin test results, both in the pilocarpine model. The percentages of time in relation to the control for the occurrence of the first salivation are presented with the administration of the following treatment doses: control (saline); hemopressin (Hp or PVNFKFLSH, 0.551334 μmol/kg); hemopressin (0.91889 μmol/kg); the peptide of the invention SEQ ID No. 1 (0.540882 μmol/kg); SEQ ID No. 1 (0.901469 μmol/kg); or PEP-19 (DIIADDEPLT, 0.908117 μmol/kg). The asterisks indicate the statistical significance: (*) P<0.05 vs Control; (**) P<0.01 vs Control; the + sign indicates P<0.05 vs Hp 0.91889 μmol/kg.

On the one hand, salivation induced by administration of pilocarpine is indicative of changes in muscarinic receptors. Accordingly, the substantial change in the time profile for the occurrence of the first salivation observed with prior administration of the compound of the invention suggests modulation, either directly or indirectly, of muscarinic receptors.

In addition, the results of FIG. 2 clearly show that oral administration of the compound of the present invention provides substantially higher salivation time modulating activity substantially greater than that observed with oral administration of Hp.

### Example 4, Comparative Anticonvulsant Pharmaceutical Composition Comprising the Compound of SEQ ID No. 1 with the Anticonvulsant Pharmaceutical Composition Comprising Hp—Results of In Vivo Tests

In this example, the anticonvulsive effects of the composition of the invention were compared to the pharmaceutical composition containing Hemopressin (Hp or PVNFKFLSH), the use of which as an anticonvulsant is co-pending patent application PCT/BR2017/050313, the same inventors.

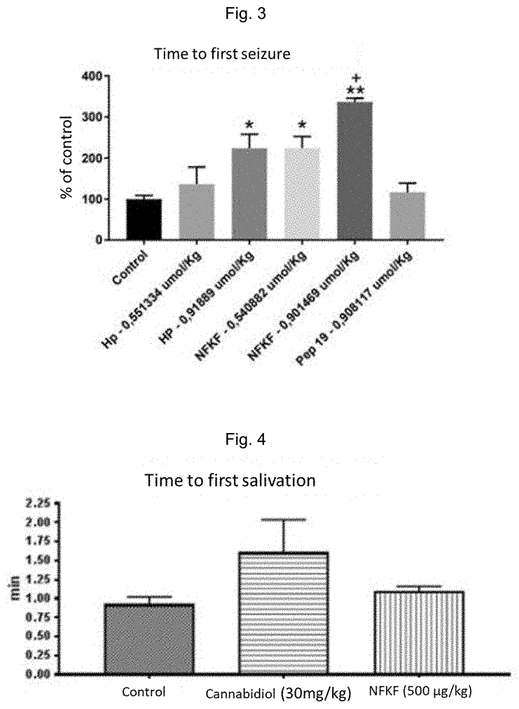

FIG. 3 shows the test results of the compound of the invention SEQ ID No. 1 as an anticonvulsant compared to the results of tests of hemopressin as an anticonvulsant, both in the pilocarpine model. The percentages of time (relative to the control) for the first seizure occur with administration of the following treatment doses: hemopressin (Hp or PVNFKFLSH, 0.551334 μmol/kg); hemopressin (0.91889 μmol/kg); the peptide of the invention SEQ ID No. 1 (0.540882 μmol/kg); SEQ ID No. 1 (0.901469 μmol/kg); PEP-19 (DIIADDEPLT, 0.908117 μmol/kg) and control (saline). The asterisks indicate: (*) P<0.05 vs Control; (**) P<0.01 vs Control; the + sign indicates P<0.05 vs Hp 0.91889 μmol/kg.

Furthermore, the results of FIG. 3 clearly show that oral administration of the compound of the present invention provides activity modulating the occurrence of the first seizure substantially greater than that observed with oral administration of Hp. For example, the same effect is observed with half the dose of the compound of the invention when compared to Hp.

### Example 5, Anticonvulsive Pharmaceutical Composition Comprising the Compound of SEQ ID No. 1—Results of In Vivo Tests

In this embodiment, the anticonvulsive effect of the composition of the invention prepared according to Example 2 was evaluated by prior administration of the inventive composition and subsequent administration of pilocarpine to animals. FIG. 4 presents the results of the pilocarpine model, indicating the time for the first salivation with the administration of control, cannabidiol (30 mg/kg), the compound of the invention R1-N-AA1-K-AA2-R2, in which embodiment is the tetrapeptide of SEQ ID No. 1 (500 μg/kg).

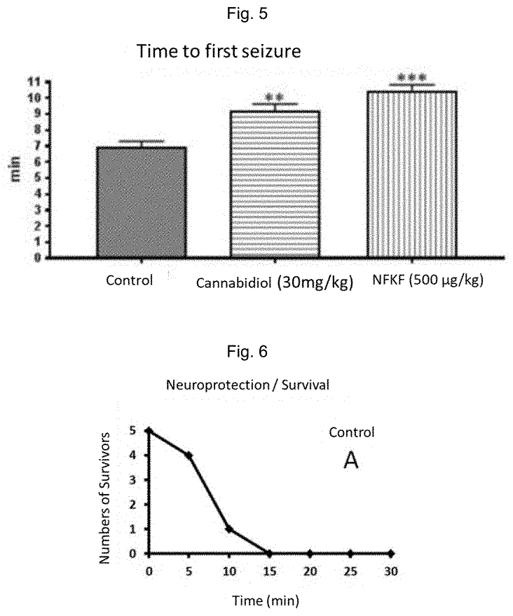

FIG. 5 presents the results of pilocarpine modeling, indicating the time for the first seizure to occur with the administration of cannabidiol (30 mg/kg) and the peptide of the invention SEQ ID No. 1 (500 μg/kg). The asterisks indicate: (**) P<0.02 vs Control; (***) P<0.002 vs Control.

In addition to the results shown in FIGS. 4 and 5, important additional technical effects include observation of no occurrence of prostration or nasal bleeding in animals undergoing treatment with the compound of the invention SEQ ID No. 1, whereas in the group of animals treated with cannabidiol both prostration and the nasal bleeding was observed. These, therefore, are additional technical problems solved by the compound of the invention.

### Example 6. Neuromodulators Pharmaceutical Composition Comprising Compound of SEQ ID No. 1—Results of In Vivo Tests

In this embodiment, the neuromodulatory effect of the composition of the invention prepared according to Example 2 was evaluated by prior administration of the inventive composition and subsequent administration of pilocarpine to animals. Other test compounds were also evaluated as described below. Administration of pilocarpine leads to severe brain injury, neurotoxicity and usually culminates in the death of the animals. This substance was used in the experiments described below but its harmful neuronal/encephalic effects were inhibited by the prior administration of the composition of the present invention: the vast majority of the animals subjected to these experiments had no symptoms related to brain lesions and survived without apparent damage, in contrast to groups of animals treated with other known substances.

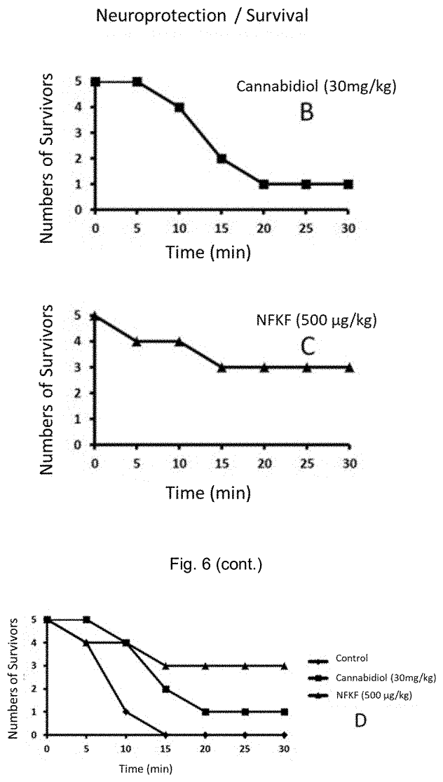

The composition of the invention prepared according to Example 2 was pre-administered to the animals. FIG. 6 shows the results of neuroprotection tests with the compound of the invention on the SEQ ID No. 1 embodiment in the pilocarpine model, the survival/death profile of the animals being indicated by the administration of the compound of SEQ ID No. 1. In A) the survival profile of the animals administered the control (saline only) is shown; In B) the survival profile of the animals administered cannabidiol 30 mg/kg is shown; In C) the survival profile of animals given the compound of SEQ ID No. 1 500 μg/kg was shown; In D) all profiles are shown in a single graph. Interestingly, in the group treated with the compound of SEQ ID No. 1 500 μg/kg only two animals died. The remaining animals in the SEQ ID No. 1 group, for a total of 3, were alive for more than a week, while practically all the others died in less than 30 minutes. Consequently, the mean survival time for this group is significantly different and much higher than the others. In addition, when comparing the SEQ ID No. 1 group (500 jjg/kg) with the group administered cannabidiol (30 mg/kg), the survival rate is three times higher in the first group, that is, in the group administered with the compound of SEQ ID No. 1 500 jjg/kg, even using a 60-fold lower relative concentration of compound.

In addition to the results shown in FIG. 6 above, important additional technical effects include observation of non-occurrence of prostration or nasal bleeding in animals undergoing SEQ ID No. 1 treatment, while in the group of animals treated with cannabidiol both prostration and bleeding were observed. These, therefore, are additional technical problems solved by the compound of the invention.

### Example 7. Neuromodulatory Pharmaceutical Composition Comprising Compound of SEQ ID No. 1, SEQ ID No. 7, or NKF—Results of In Vivo Tests

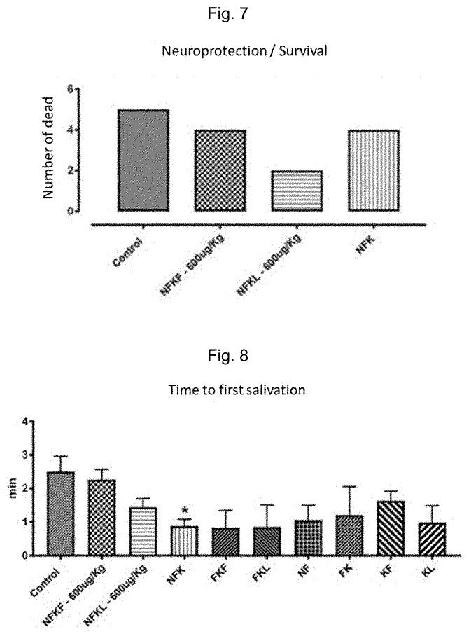

In view of the surprising results obtained in Example 6, the peptides of the invention in the SEQ ID No. 1, SEQ ID No. 7 and NKF embodiments were compared in the experiment described below, which shows that the harmful neuronal/encephalic effects of pilocarpine were inhibited by the prior administration of these embodiments of compounds of the present invention: some animals subjected to these experiments had no symptoms related to brain lesions and survived without apparent damage, in contrast to the group of animals treated with the control. The composition of the invention was prepared according to Example 2 with due concentration correction, for comparison of the effects of SEQ ID No. 1, SEQ ID No. 7 and NFK, such compounds having been previously administered to the animal groups. FIG. 7 shows the results of neuroprotection tests in the pilocarpine model, the survival/death profile of the animals being indicated by administration of these compounds of the invention and subsequently to pilocarpine. It is interesting to note that in the group treated with the compound of SEQ ID No. 7 600 μg/kg the results were even better than those obtained with SEQ ID No. 1, whereas the results with NFK were equivalent to those obtained with SEQ ID No. 1.

### Example 8. An Anticonvulsant Pharmaceutical Composition Comprising the Compounds of SEQ ID No. 1, SEQ ID No. 7, NFK, FKF, FKL—Results of In Vivo Tests

In this embodiment, the anticonvulsant effect of different embodiments of the compound of the invention with the structure represented by SEQ ID No. 1, SEQ ID No. 7, NFK, FKF, FKL, as well as the NF, FK, KF and KL dipeptides were evaluated in the pilocarpine model.

Table 1 shows the time data of occurrence of the first salivation in the model tested.

TABLE 1

| | | SEQ | SEQ | | | | | | | |
|---|---|---|---|---|---|---|---|---|---|---|
| | | ID No. | ID No. | | | | | | | |
| | control | 1 | 7 | NFK | FKF | FKL | NF | FK | KF | KL |
| 1 | 0.6667 | 1.83 | 1.183 | 1.38 | 0.38 | 1.6 | 0.83 | 0.1833 | 1.38 | 0.95 |
| 2 | 3.2000 | 1.97 | 1.067 | 0.75 | 0.95 | 0.2 | 0.97 | 1.5 | 1.75 | 0.50 |
| 3 | 1.6333 | 2.73 | 1.25 | 0.42 | 0.50 | 0.633 | 1.73 | 0.95 | 1.42 | 1.50 |
| 4 | 2.6667 | 1.58 | 2.45 | 0.98 | 1.50 | 1.5 | 0.58 | 2.183 | 1.98 | |
| 5 | 0.8333 | 3.2 | 1.25 | | | 0.333 | 1.2 | | | |

FIG. 8 presents the test results of the compounds of the invention SEQ ID No. 1, SEQ ID No. 7, NFK, FKF, FKL, as well as the dipeptides NF, FK, KF and KL in the pilocarpine model, indicating the time for the occurrence of the first salivation after administration of control or the test compounds. It is noteworthy the result of the administration of NFK, which inhibited or delayed the occurrence of first salivation in the pilocarpine model. This result indicates the modulation of muscarinic receptor function, such as acetylcholine (mAChRs), which plays an important role in cognitive functions, such as learning and memory, control of dopamine release, modulation of locomotor activity. Modulation of these receptors is useful in the control of Alzheimer's disease and/or control of dependence or addiction of drugs of abuse, as well as in protection against dementia of old age.

Table 2 shows the time data of occurrence of the first signal in the model tested.

TABLE 2

| | | SEQ | SEQ | | | | | | | |
|---|---|---|---|---|---|---|---|---|---|---|
| | | ID No. | ID No. | | | | | | | |
| | control | 1 | 7 | NFK | FKF | FKL | NF | FK | KF | KL |
| 1 | 2.67 | 8.43 | 9.317 | 6.28 | 5.3 | 5.67 | 5.67 | 6.5 | 5.8 | 8.5 |
| 2 | 10.2 | 10.23 | 10.17 | 8.92 | 6.35 | 9.5 | 4.99 | 7.33 | 6.33 | 7.33 |
| 3 | 10.5 | 13.53 | 3.83 | 4.83 | 8.33 | 8.5 | 5.5 | 7.4 | 6.5 | |
| 4 | 5.5 | 9.9 | 10.17 | 7.82 | 8.82 | 4.5 | 3.5 | 6.17 | 7.5 | |
| 5 | 9.65 | 12.33 | 10.25 | | | 9.17 | 6.5 | | | |

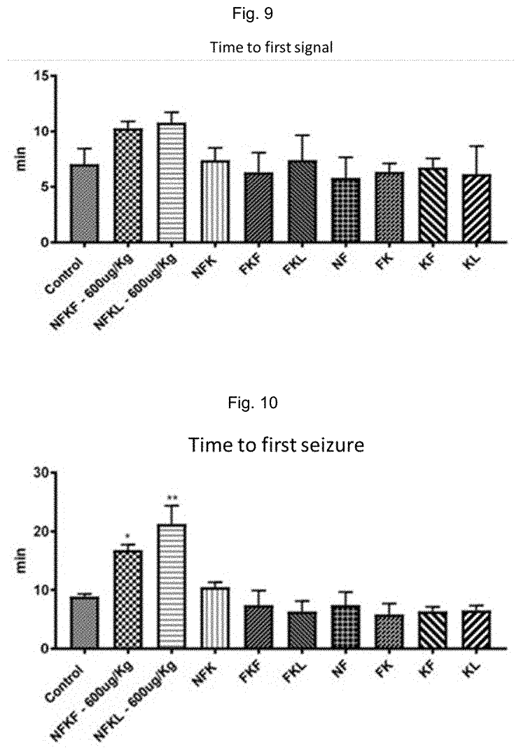

FIG. 9 shows the test results of the compounds of the invention SEQ ID No. 1, SEQ ID No. 7, NFK, FKF, FKL, as well as the dipeptides NF, FK, KF and KL in the pilocarpine model, indicating the time for the occurrence of the first signal after control administration or the test compounds.

Table 3 shows the time data of occurrence of the first seizure in the model tested.

TABLE 3

| | | SEQ | SEQ | | | | | | | |
|---|---|---|---|---|---|---|---|---|---|---|
| | | ID No. | ID No. | | | | | | | |
| | control | 1 | 7 | NFK | FKF | FKL | NF | FK | KF | KL |
| 1 | 7.9 | 15.17 | 18.4 | 11.33 | 6.28 | 5.3 | 5.67 | 5.67 | 6.5 | 5.8 |
| 2 | 10.5* | 19.95 | 15.33 | 9.67 | 8.92 | 6.35 | 9.5 | 4.99 | 7.33 | 6.33 |
| 3 | 8.38 | 15.18 | 30 | 8 | 3.83 | 4.83 | 8.33 | 8.5 | 5.5 | 7.4 |
| 4 | 9.55 | 16 | 27.3 | 9.93 | 7.82 | 8.82 | 4.5 | 3.5 | 6.17 | |
| 5 | 9.65 | 17.88 | 15.33 | 13.38 | 10.25 | | 9.17 | 6.5 | | |

FIG. 10 shows the test results of the compounds of the invention SEQ ID No. 1, SEQ ID No. 7, NFK, FKF, FKL, as well as the dipeptides NF, FK, KF and KL in the pilocarpine model, indicating the time for the occurrence of the first seizure after control administration or the test compounds.

Table 4 shows the time data of death occurrence in the model tested.

TABLE 4

| | | SEQ | SEQ | | | | | | | |
|---|---|---|---|---|---|---|---|---|---|---|
| | | ID No. | ID No. | | | | | | | |
| | control | 1 | 7 | NFK | FKF | FKL | NF | FK | KF | KL |
| 1 | 13.17 | 18.92 | 26.63 | 13.53 | 13.17 | 14.53 | 13.17 | 13.17 | 11.7 | 16.67 |
| 2 | 18.67 | 20 | 25.13 | 11.7 | 20.67 | 11.7 | 20.67 | 20.67 | 10.92 | 13.5 |
| 3 | 17.67 | 15.18 | | 10.92 | 16.67 | 10.92 | 16.67 | 16.67 | 13.5 | 19.88 |
| 4 | | 10.87 | 13.5 | 10.87 | 13.5 | | | | | |
| 5 | 13.9 | | | 16.83 | 12.9 | | 12.9 | | | |

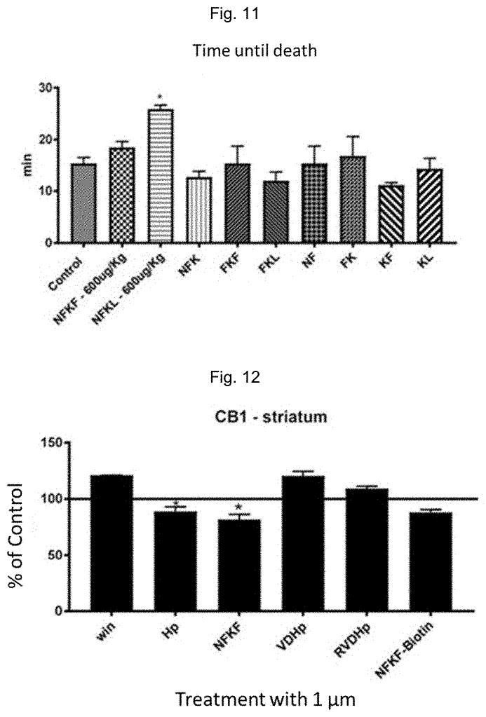

FIG. 11 shows the test results of the compounds of the invention SEQ ID No. 1, SEQ ID No. 7, NFK, FKF, FKL, as well as the dipeptides NF, FK, KF and KL in the pilocarpine model, indicating the time for the occurrence of death after administration control compounds or test compounds.

In the cases of FKF, FKL, NF, FK, KF and KL tests from Tables 1-4 above, blank fields are from animals that convulsed and died at the same time.

The results of the tests performed in Examples 3 to 8 above show significant and significant in vivo results in dosage ranges of the order of 500 to 1000 μg of compound of the invention per kg of animal. Considering the tests in mice and the conversion to the Human Equivalent Dose mentioned above, effects of the same magnitude are expected in humans in the dosage range of 40 to 80 μg of compound of the invention per kg of human and, Safety ranges, concentrations between 4 to 800 μg/kg for administration to humans are considered in the present invention.

### Example 9. Use of the Compound R1-N-AA1-K-AA2-R2 as the Synthesis Intermediate in the Preparation of Other Compounds

In the present application, the peptidic compound of the invention is also useful as a synthetic intermediate in obtaining other compounds of pharmaceutical/diagnostic interest. In this and other embodiments the compound is referred to as a “modified peptide”, which is unnatural, being artificially modified or obtained by synthesis, including halides, cyclized, amidated, alkylated, alkoxylated, hydroxylated, PEGylated, other functional groups in any amino acids, or salt forms thereof, as well as modified with an amino acid or peptide, including the non-natural as the d-amino acid forms. In the embodiment of this example, the peptidic compound of the invention has been modified with molecular species useful in diagnostic and/or therapeutic applications, such as Biotin, using techniques known to those skilled in the art. Biotin is a label or tag that can be used in the detection of the ligand target of the invention, in the present case the cannabinoid and/or muscarinic receptors, through the use of anti-biotin antibodies, or avidin/streptavidin detection strategies with enzymes such as horseradish peroxidase, alkaline phosphatase, or fluorescent probes.

The modified compound of the invention in the biotinylated embodiment was used in Screening techniques using anti-CB1 receptor antibodies sensitive to conformational changes.

Antibodies sensitive to CB1 receptor activation were generated and characterized by their specificities. For screening compounds, Striatum membranes (5 μg per well) were plated on NUNC-Immuno 96-well plates (Nalge-Nunc) and dried at room temperature. Membranes were washed with PBS (phosphate buffered saline) and incubated with or without different ligands (1 μM) and the extent of receptor recognition by the antibodies was measured by ELISA. The results of the tests are shown in Table 5, which indicates the % binding to the CB1 receptor in relation to control:

TABLE 5

| | Assay | | | | |
|---|---|---|---|---|---|
| Compound | 1 | 2 | 3 | 4 | 5 |
| Win | 111.483 | 117.95 | 125.56 | 127.52 | 122.49 |
| Hp | 91.33 | 92.74 | 97.05 | 90.23 | 73.49 |
| SEQ ID No. 1 | 90.27 | 84.41 | 90.19 | 77.88 | 65.39 |
| VDHp | 115.72 | 113.62 | 131.029 | 128.99 | 113.05 |
| RVDHp | 103.39 | 111.53 | 110.87 | 114.76 | 105.22 |
| SEQ ID No. 1- | 89.23 | 85.55 | 83.29 | 84.76 | 97.47 |
| Biotin | | | | | |

FIG. 12 shows these results and confirms that the peptidic compound of the invention has also been shown to be an appropriate synthetic intermediate for the preparation of other compounds useful in diagnostic and/or therapeutic applications. The peptide compound of the invention, when modified/protected by biotin, has also been shown to bind to the CB1 receptor in tests with anti-CB1 antibodies sensitive to the activation/conformational changes of the receptor.

In this context, the present co-inventor has already shown (Gupta et al.) that μ and δ opioid receptors dimerize with CB1 cannabinoid receptors and that heterodimerization leads to modulation of receptor function. In addition, the use of other CB1 receptor binding substances provides increased recognition of μ opioid receptors, apparently mediated by conformational changes in them. These observations support the idea that conformational changes caused by CB receptor ligands may affect the receptor partner in a heterodimer and that this can be detected using antibodies sensitive to conformational changes. Thus, the test results in this example support the concept of an in vitro diagnostic process for protein-protein or peptide-protein interactions that modulate the function of GPCRs (G protein coupled receptors, or G-Protein Coupled Receptors/GPCRs), including heterodimeric interactions.

The results of the present example support one of the invention objects, which is an in vitro process for diagnosing the presence, quantity and/or localization of cannabinoid, opioid and/or muscarinic receptors, as well as evaluating the binding of other compounds to these receptors.

Said process comprises contacting the optionally modified compound of the invention with a biological sample containing a cannabinoid, opioid and/or muscarinic receptor. Said method is also useful in the identification and/or quantification of other molecular entities binding to these receptors, and includes a step of detecting said visual, optical, radioactive, chemical, spectroscopic signal connection. Examples include biochemical, cytochemical, histochemical tests. The ligand of the invention is also potentially useful for in vivo diagnostic and/or prognostic processes, including contrast and non-contrast imaging tests, for example magnetic resonance spectroscopy (MRS) of molecular entities whose presence and/or amount is modulated by administration of the compound of the invention. This approach is potentially useful in the diagnosis and/or prognosis of degenerative diseases, especially neurodegenerative diseases.

### Example 10. In Vitro and in Silico Binding/Interaction Tests of the Compound SEQ ID No. 1 to the CB1 Receptor

In this example, the in vitro affinity profile of the compound of the invention R1-N-AA1-K-AA2-R2, in which is the SEQ ID No. 1, was evaluated with the cannabinoid receptor CB1.

For this purpose, binding techniques were used to measure the affinity of the SEQ ID No. 1 (powder in 100% purity and prepared as stock solution 10 mM in DMSO) by the cannabinoid CB1 receptor at concentrations of 1 and 10 μM. This evaluation was done using the human recombinant CB1 cannabinoid receptor, associated with the radioligand agonist CP 55940 (IC 50 (M)=1.1 nM; Ki (M)=0.94 nM; nH=1). In vitro binding assays were performed using recombinant CHO cells expressing the human CB1 receptor and the [3H] CP 55940 linker (0.5 nM concentration; 3.5 dM Kd) in incubation for 120 minutes at 37° C. using the compound not specific WIN 55212-2 (10 mM), with detection by scyntilography counting (Munro et al., 1993).

Results are expressed as the percentage of binding of the specific control according to formula:

$$\frac{Binding specific as measured}{Binding specific of control} \times 100$$

And the percentual inhibition of binding of specific control obtained in the presence of the compound of the invention is:

$$100 - ( \frac{Binding specific as measured}{Binding specific of control} \times 100 )$$

IC50 values (concentration that causes half of maximal inhibition of specific control binding) and Hill (nH) coefficients were determined by nonlinear regression of the competition curves generated with the mean values of the replicates using the Hill equation:

$$Y = D + ( \frac{A - D}{1 + {( C / {C}_{50} )}^{nH}} )$$

Where Y=specific binding, A=asymptotic left of curve, D=asymptotic right of curve, C=compound concentration, C₅₀=IC₅₀ and nH=curve coefficient/slope factor. This analysis was performed using the Hill software validated by comparison with data generated by Sigmaplot 4.0 software. Inhibition constants (Ki) were calculated using the equation of Chen Prusoff:

$${K}_{i} = \frac{{IC}_{50}}{1 + L / {K}_{D}}$$

Where L=concentration of the radioligand in the assay; K_{D}=affinity of the radioligand for the receptor. A graph is used to determine the K_{D}.

Results showing inhibition or stimulation greater than 50% are considered to represent significant effect of the test compound. Results showing inhibition or stimulation between 25% and 50% are indicative of low to moderate effect of the test compound. Results showing inhibition or stimulation of less than 25% can be considered as minor. Results showing inhibition greater than or equal to 50% are indicative non-specific or allosteric effects of the test compound.

The results obtained in duplicate for each concentration tested (1 and 10 μM) are shown in Table 6.

TABLE 6
In vitro inhibition data of binding of the radioligand agonist
CP55940 to the CB1 receptor by SEQ ID No. 1

| | 1st | 2nd | Mean |
|---|---|---|---|
| Concentration | measurement | measurement | value |
| 1 mM | −121.4 | −27.8 | −74.6 |
| 10 mM | −18.9 | −0.9 | −9.9 |

% of inhibition of binding of specific control

Even if the first measurement were disregarded at 1 mM, due to the surprisingly high value, the data of the second measurement at 1 mM taken alone would still be considered indicative of effect.

The results indicate that SEQ ID No. 1 has greater power to modulate the binding of the respective radioligand when at low concentrations, suggesting that in the range of nM the effects are greater.

These results indicate that the likely mechanism of action associated with the therapeutic profile evidenced for the SEQ ID No. 1 involves allosteric modulation of CB1 cannabinoid receptors, which is consistent with the astonishing/unexpected in vivo test results (Examples 3, 4, 5, 6, 7, 8), as well as the results of the in vitro experiments described above and the in silico experiments described below.

These experimental data indicate that the compound of the invention interacts with and/or modulates the activity of the CB1 receptor and/or its endogenous ligands. Thus the compound of the invention is potentially useful in various metabolic functions, including control of food intake, metabolism of energy and/or lipids, regulation of intestinal motility, immune system, calcium cycle balance, among others. Cannabinoid receptors are widely expressed in the brain, including cortex, hippocampus, amygdala, pituitary, hypothalamus, adrenal gland. CB receptors, particularly CB1, have already been identified in numerous peripheral organs and tissues, including the thyroid gland, reproductive organs, adipose tissue, liver, muscle, and gastrointestinal tract. These results support the use of the compound of the invention in the modulation of metabolic functions and/or neuromodulation.

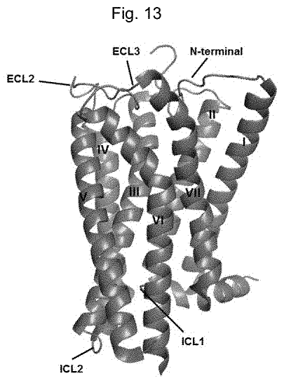

The cannabinoid receptor 1 (CB1) corresponds to the GPCR (Expressed Receptors to Protein G) most expressed in the human brain and is found at high levels in the Central Nervous System in general (FIG. 13), It is activated by endocannabinoids and has been identified as a promising therapeutic target for the treatment of various diseases such as pain and inflammation, multiple sclerosis and neurodegenerative diseases (agonist effect) and obesity, liver fibrosis and nicotine dependence (HUA) et aL, 2016).

FIG. 13 shows an overview of the three-dimensional structure of a GPCR, in this case, the cannabinoid receptor of subtype 1. The seven transmembrane helices (I-VII), the intracellular loops (ICL1 and ICL2) and the extracellular loops ECL2 and ECL3).

Through the use of Molecular Modeling tools we have evaluated in this example the mode of interaction at the CB1 binding site of the compound of the invention R1-N-AA1-K-AA2-R2, in which embodiment it is the SEQ ID No. 1, In addition, the profile observed was compared with the mode of interaction of other known binders, such as cannabidiol, rimonabant and AM6538.

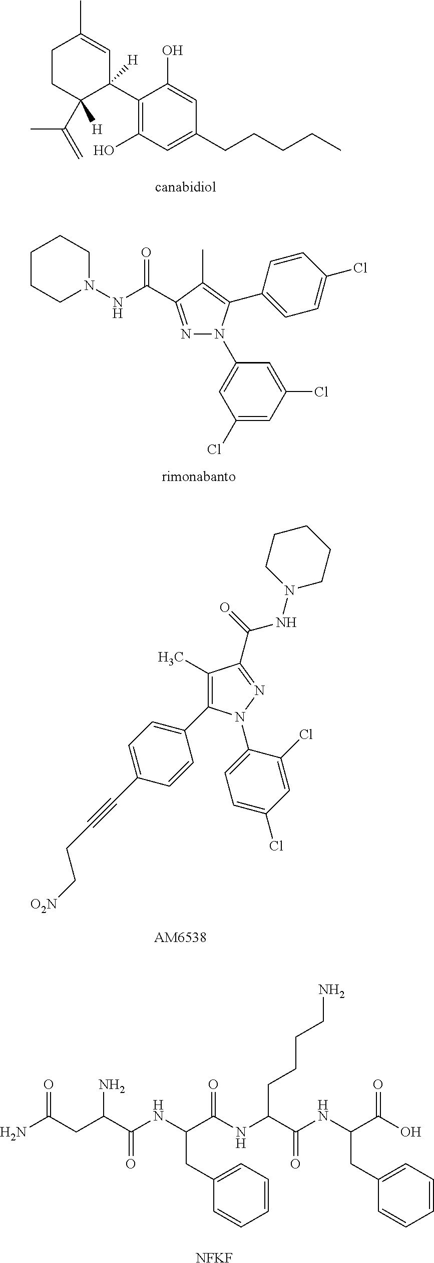

The in silico tests of the interaction profile of the SEQ ID No. 1 with the CB1 receptor were made from the design and optimization of the ligand structures. Initially, the Percepta program was used to verify the ionization status of these ligands at plasma pH (pH=7.4). Subsequently, the construction of ligand structures was performed in the Spartan v. 16 (Wavefunction, Inc.) followed by energy minimization by molecular mechanics.

The selection of the crystallographic structure CB1 for the docking studies was made from the data available in the PDB code 5TGZ, belonging to the species Homo sapiens and presenting a resolution of 2.8 Å. Said structure corresponds to the only available CB1 crystal in the database and available at the priority date of this patent application.

The validation of the functions of the GOLD v. 5.1 and conducting docking studies. To identify the most stable complex, three scoring functions were considered in the GOLD Program (ChemScore, Goldscore and ChemPLP), For the realization of molecular anchoring in the GOLD v. 5.5 (CCDC) the location of the active site was required using the co-crystal I ized binder (AM6538) itself and all amino acids at 6 Å distance thereof as reference. Subsequently, molecular anchoring and analysis of the results in the PyMOL v. 1.8.6.2 (SchrÖdinger, LCC).

Initially, the scoring functions of the GOLD v. 5.5 were evaluated according to the ability to predict protein-binding complexes compatible with experimental results. For this, three scoring functions (Goldscore, Chemscore, and ChemPLP) were validated by redocking the reference ligand, AM6538, at the CB1 receptor binding site (PDB 5TGZ). Firstly, docking was performed in the absence of water molecules and, afterwards, trying to estimate how much the water molecules present in the binding site could influence the orientation of the binder, the same evaluation was made in the presence of water molecules. The results obtained by Root Mean Square Deviation RMSD, considering the complexes with higher scores, can be observed in Table 7.

For the calculation of the RMSD are considered and compared pairs of atoms of the result of the molecular modeling experiments and the experiments of crystallographic structure. Thus, the square root of the mean between the distances of these atoms is calculated, obtaining the values for the deviation. The smaller the deviation, the greater the approximation between the results, indicating, therefore, the most adequate function to be used (Maiorov; Crippen, 1994; Hildebrandt et ai., 2013).

TABLE 7
RMSD values obtained from the validation of the scoring functions of
the GOLD v. 5.5 for the redocking of AM6538 in the CB1 receptor.

| | RMSD | |
|---|---|---|
| Function | with water | w/o water |
| CHEMPLP | 0.9543 | 0.9134 |
| CHEMSCORE | 1.1762 | 1.1578 |
| GOLDSCORE | 0.9384 | 0.8864 |

As indicated in Table 7, the function which showed the lowest RMSD values, and which proved to be most suitable for the docking experiments, was Goldscore in the absence of water molecules at the binding site, indicating that such molecules do not influence the mode of the ligand.

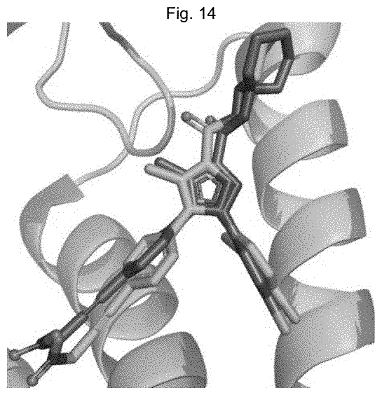

FIG. 14 shows visually the overlap between the crystallographic structure complex (PDB 5TGZ) and the resulting complex from the AM6538 redocking experiment using the validated methodology.

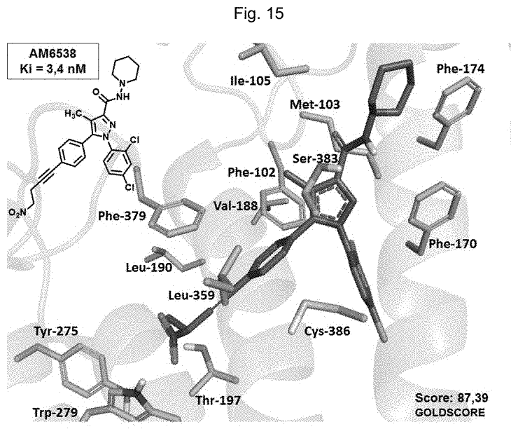

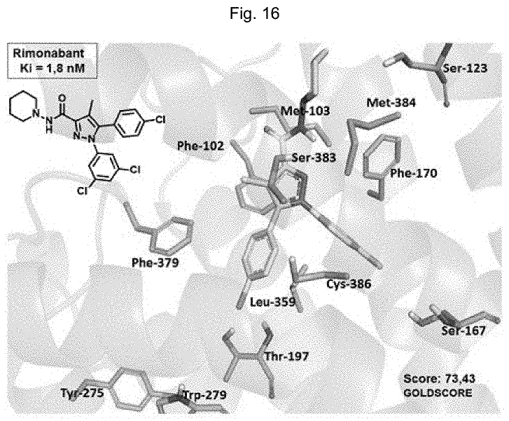

The complex resulting from the redocking of AM6538 in CB1 obtained a score of 87.39 and the interaction profile observed was consistent with that described in the literature (Hua et al., 2016), with several hydrophobic interactions with amino acid residues such as: Phe-102, Phe-170, Phe-174 and Phe-379, as well as leucine, methionine, cysteine and valine residues (see FIG. 15).

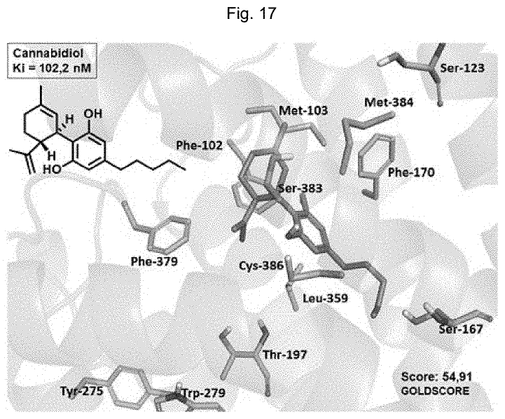

On the other hand, for the rimonabant and cannabidiol, the interaction profiles observed were very similar to the AM6538, respectively, presenting scores of 73.43 and 54.91 (FIGS. 16 and 17, respectively).

In the docking experiment of one embodiment of the compound of the invention (SEQ ID No. 1) its structure was analyzed for its ionizing state at plasma pH (pH=7.4) in the Percepta program, which indicated the structure shown below, which was used for docking studies

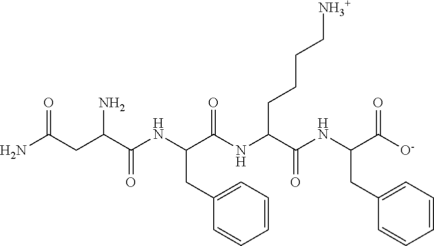 [SMILES: NC(=O)CC(N)C(=O)NC(Cc1ccccc1)C(=O)NC(CCCC[NH3+])C(=O)NC(Cc1ccccc1)C(=O)[O-]]

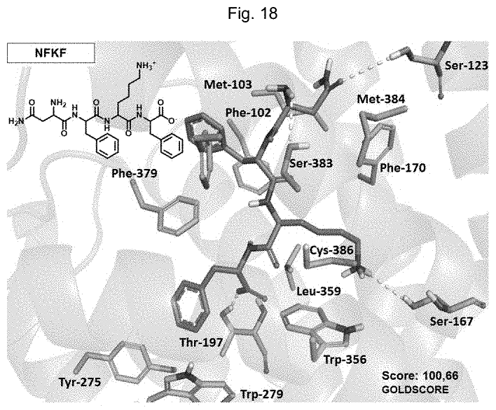

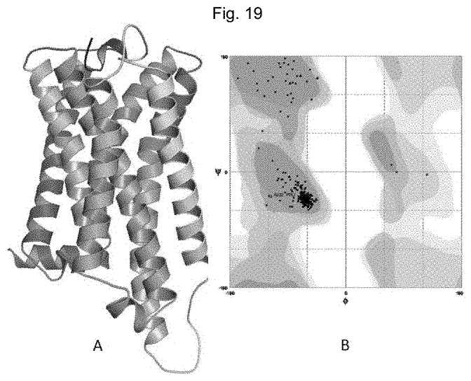

The resulting docking complex of the SEQ ID No. 1 in CB1 scored 100.66, substantially higher than the score achieved by the reference ligands. This fact can be explained by additional hydrogen bonds observed between the tetrapeptide and the residues Ser-123, Thr-197, Ser-167 and Ser-383, In addition, the hydrophobic interactions observed for AM6538 are also present in the SEQ ID No. 1 interaction mode (FIG. 18).

Together, the results of these experiments show that the observed interaction profile for SEQ ID No. 1, with similar interactions to the reference ligands, as well as additional interactions that resulted in a higher score obtained in the docking studies, suggest that the SEQ ID No. 1 is a potential ligand of cannabinoid receptors of subtype 1.

### Example 11. In Vitro and in Silico Binding/Interaction Tests of Compound SEQ ID No. 1 to CB2 Receptor

In this example, the in vitro affinity profile of the compound of the invention R1-N-AA1-K-AA2-R2, in which is the SEQ ID No. 1, was evaluated with the cannabinoid receptor CB2.

For this purpose, binding techniques were used to measure the affinity of the SEQ ID No. 1 (powder in 100% purity and prepared as stock solution 10 mM in DMSO) by the cannabinoid receptor CB2 at concentrations of 1 and 10 μM. This evaluation was done using the recombinant human CB2 cannabinoid receptor associated with the radioligand agonist WIN 55212-2 (IC 50 (M)=1.5 nM; Ki (M)=0.96 nM; nH=0.9). In vitro binding assays were performed using recombinant CHO cells expressing human CB2 receptor and [3H] WIN55212-2 linker (0.8 nM concentration; 1.5 nM Kd) in incubation for 120 minutes at 37° C. using non-specific compound WIN 55212-2 (5 mM), with detection by scyntilography counting (Rinaldi-Carmona et al, 1996).

The results are expressed as indicated in Example 10 above.

The results obtained in duplicate for each concentration tested (1 and 10 jjM) are shown in Table 8.

TABLE 8
In vitro inhibition data of binding of radioligand agonist
WIN 55212-2 to CB2 receptor by SEQ ID No. 1

| | 1st | 2nd | Mean |
|---|---|---|---|
| Concentration | measurement | measurement | value |
| 1 mM | 3.5 | 0.3 | 1.9 |
| 10 mM | 9.5 | 13.1 | 11.3 |

i. % of inhibition of binding of specific control

The results indicate that SEQ ID No. 1 did not provide significant modulation in binding of the respective radioligand under the conditions tested.

Taking these results together with the results of the experiments described in Example 10, one arrived at interesting conclusions. Although the SEQ ID No. 1 peptide has demonstrated a selective ligand of CB2 cannabinoid receptors, as indicated by the results of the docking experiments for this receptor, in vitro test results indicate that this tetrapeptide is not able to bind to this cannabinoid receptor in a significant manner. However, an interaction of the compound of the invention with the allosteric type CB2 receptor cannot be ruled out. Thus, the test results indicated a positive modulation of CB2 receptor, although moderate at the concentrations tested, suggest a combined (allosteric) modulation effect which is negative for CB1 and positive for CB2.

In addition, in vitro test results (Examples 9, 10 and/or 11) demonstrate that the compound of the invention can be used as a binder of diagnostic interest, such as, for example, radioactive or chromophore labeling for subsequent identification of binding sites in cells and/or tissues.

In order to enable in silico experiments of the interaction of the compound of the invention R1-N-AA1-K-AA2-R2, in which it is the SEQ ID No. 1, with the CB2 receptor, a three-dimensional model of the CB2 receptor was first constructed, for subsequent docking experiments.

In this embodiment, the construction of the 3D model of CB2 was done from a search in the UniProt database of the amino acid sequence of this receptor. The criterion used for sequence selection was the species (Homo sapiens). Thus, the sequence selected for carrying out the molecular modeling studies was that of code P34972.

Next, the Template Identification tool of the SwissModel server was used to identify and select the protein template. The sequence identified by the server as having the highest structural identity to the human CB2 sequence was that of PDT 5TGZ code (Hua et al., 2016) belonging to the species Homo sapiens, corresponding to the CB1 receptor.

The target and template sequences were then aligned using the ClustalW2 software linked to the UniProt database to compare the sequences to establish the percentage of identity between them. The 3D homology model of human CB2 was constructed using the Automated Mode tool available on the Swiss-model server page and, for validation of the generated model, the overall structural quality and stereochemical quality of the model were analyzed through the value presented for the GMQE parameter and the Ramachandran graph analysis.

After the creation of the 3D model for CB2, docking studies were performed for the SEQ ID No. 1 as well as for cannabidiol, rimonabant and AM6538.

After identification and selection of the human CB2 receptor sequence in UniProt and the identification of the template protein (PDB code 5TGZ) from the Template Identification tool on the Swiss-Model server, alignment was made between the two sequences in the program ClustalW2. The identity between the two sequences was 46.18%, which makes possible the use of comparative modeling as a tool to obtain the 3D structure of the human CB2 receptor, as recommended in previously published studies (Morgan and Hurley, 2015), which affirm be necessary the identity of at least 30% between primary sequences for the creation of homology models.

The stereochemical quality of the model was analyzed using the Ramachandran graph (FIG. 19B). The Ramachandran graph corresponds to a mathematical model, which relates the dihedral angles ϕ (angle C—N—Cα-C) on the x-axis and ψ (angle N—Cα-C—N) on the y-axis. This graph is divided into regions capable of representing the probability of a combination between the angles ϕ and ψ (Ramachandran; Ramakrishnan; Sasisekharan, 1963). These regions are classified as: favorable, permitted, generously permitted and prohibited.

The Ramachandran graph constructed for the human CB2 model showed the presence of approximately 96% of the amino acid residues in the most favorable regions and more than 4% of the residues in acceptable regions. No amino acid residue was found in forbidden regions, indicating the stereochemical validation of the model created. After the validation of the model, docking studies on CB2 of the SEQ ID No. 1, the AM6538 antagonist, the cannabidiol endogenous ligand and the rimonabant antagonist were performed.

For all compounds analyzed, the scores obtained in the studies were lower than those achieved for CB1, from 61.53 for AM6538, 72.33 for SEQ ID No. 1, 43.31 for cannabidiol, and finally 57.40 for rimonabant.

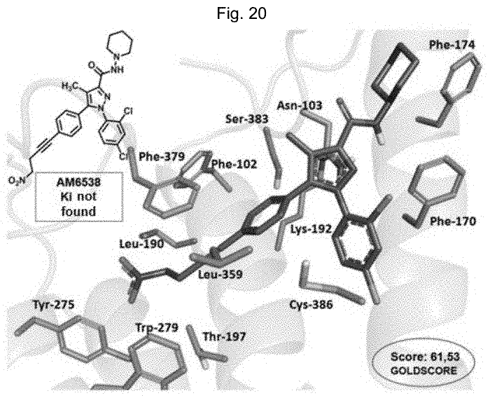

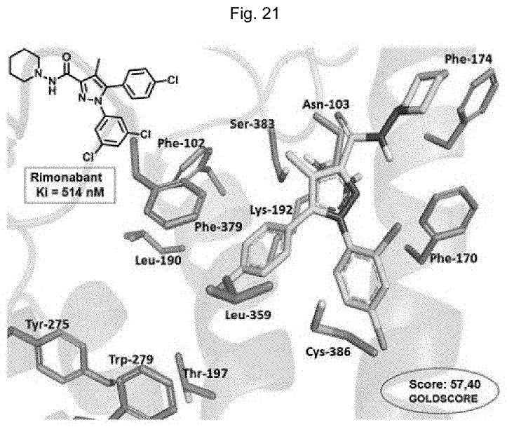

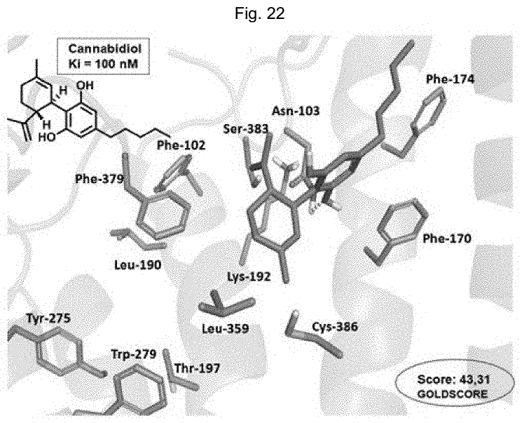

Since, in a simplistic way, punctuation values can be considered as a sum of the interactions identified between the protein and the linker, the lower scores can then be explained by a lower number of interactions at the binding site, although the major hydrophobic interactions observed in CB1 are maintained in CB2 as can be seen in FIGS. 20-23.

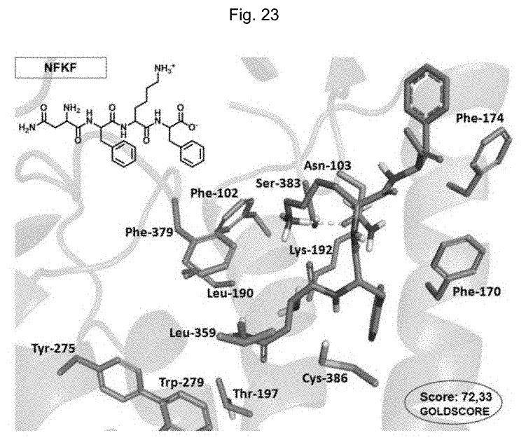

It is also important to note that for the SEQ ID No. 1 the hydrogen bonding interactions indicated in CB1 do not occur in CB2, contributing to its lower CB2 score (FIG. 23), although this peptide presented another hydrogen bond with the Lys-192 residue.

Table 9 briefly summarizes the comparison between the scores obtained by the docking studies in both receivers (CB1 and CB2) in Example 10 and in this example.

TABLE 9
Comparison between the scores obtained in the docking studies
for CB1 and CB2 receptors.

| | Scores | |
|---|---|---|
| Ligands | CB1 | CB2 |
| Cannabidiol | 54.91 | 43.31 |
| Rimonabant | 73.43 | 57.40 |
| AM6538 | 87.39 | 61.53 |
| SEQ ID No. 1 | 100.66 | 72.33 |

The results of the experimental tests described in Examples 9, 10 and/or 11 above allowed the replication, for the other compounds tested, of interaction mode of antagonist AM6538 at the CB1 receptor binding site, as described in the literature by Hua et al. In addition, the results provided validation of the docking methodology for experiments with SEQ ID No. 1 compared to cannabidiol and rimonabant antagonist, and it is therefore possible to predict their interaction profiles in CB1.

Construction and validation of a 3D model by homology of the CB2 receptor also resulted in visualization of the behavior of all of the ligands mentioned at the binding site of such receptor, providing a comparative analysis with results obtained with CB1.

In summary, the results of the in vitro and in silico experiments of Examples 9, 10 and 11 indicate that the compounds of the invention are CB1 receptor ligands. In addition, less favourable interactions of the SEQ ID No. 1 compound were evidenced in CB2 receptors, indicating a possible selectivity profile. The results of the experiments of Examples 10 and 11, notably those shown in FIGS. 17 and 23, show that NFK amino acids participate much more strongly in the binding of CB1 and CB2 than the amino acid F in the C-terminal position, indicating that this tripeptide is a potential candidate for ligand/modulator of such receptors. The results of the in vivo tests shown in FIG. 7 are in line with this statement.

### Example 12. Analgesic or Neuropathic Pain Modulating Pharmaceutical Composition Comprising the Compound of the Invention—Results of In Vivo Tests

The compounds of the present invention have also been tested as analgesics or neuropathic pain modulators. To that end, the compounds PVNFKF, PVNFK, VNFKF, SEQ ID No. 1 were tested in the pain threshold model. The pain threshold was measured using a pressure apparatus (Ugo Basile, Italy), essentially as described (Randall and Selitto, 1957). Briefly, a force of increasing magnitude (16 g/s) was applied to the back of the right paw of rats. When animals react by kicking, the force in grams required to induce this response is the threshold of pain. Antinoniceptive activity was expressed as the increase in pain threshold compared to control animals.

This induced pain model does not require concomitant administration of other substances. The compounds of the invention were administered orally to the animals at doses of 0.5 to 0.25 mg/kg and saline was used as a control.

Table 10 shows the strength results in grams for pain threshold:

TABLE 10
Immediate

| | | | | | | | |
|---|---|---|---|---|---|---|---|
| 1 | Control | 70 | 70 | 60 | 65 | 70 | 70 |
| 2 | PVNFKF | 75 | 65 | 60 | 70 | 80 | 75 |
| 3 | PVNFK | 70 | 75 | 75 | 65 | 60 | 75 |
| 4 | VNFKF | 70 | 65 | 65 | 70 | 75 | 75 |
| 5 | SEQ ID | 70 | 75 | 75 | 75 | 80 | 60 |
| | No. 1 | | | | | | |

After 3 h

| | | | | | | | |
|---|---|---|---|---|---|---|---|
| 1 | Control | 30 | 55 | 55 | 50 | 45 | 40 |
| 2 | PVNFKF | 45 | 30 | 55 | 45 | 65 | 55 |
| 3 | PVNFK | 55 | 45 | 40 | 55 | 60 | 40 |
| 4 | VNFKF | 85 | 40 | 35 | 50 | 50 | 50 |
| 5 | SEQ ID | 80 | 70 | 80 | 75 | 70 | 70 |
| | No. 1 | | | | | | |

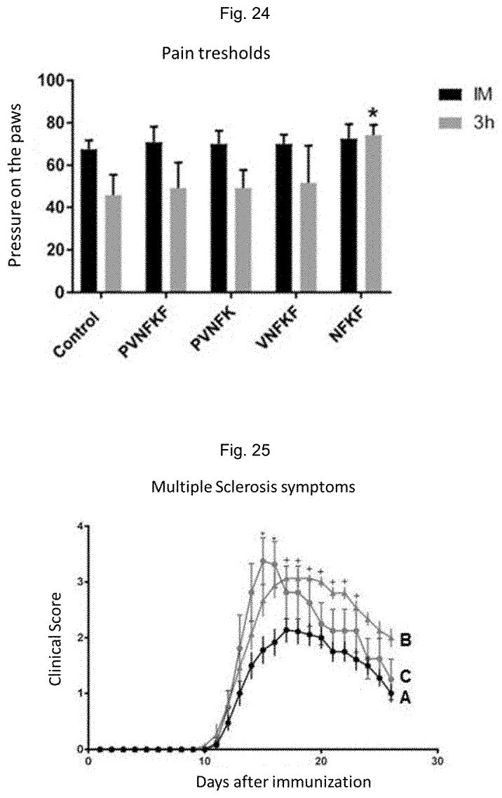

FIG. 24 shows the pain threshold results, force in grams applied to the back of the right paw of the animals. Significant and very evident is the analgesic effect provided by compound of SEQ ID No. 1, which increased the pain threshold after 3 h of administration when compared to the immediate pain threshold. In addition, compound of SEQ ID No. 1 was shown to be significantly and substantially superior to PVNFKF, with a 50.1% greater pain threshold in animals administered SEQ ID No. 1 compared to PVNFKF.

In this context, the present co-inventor has already demonstrated (Gupta et al) that μ and δ opioid receptors dimerize with CB1 cannabinoid receptors and that heterodimerization leads to modulation of receptor function. In addition, the use of other CB1 receptor binding substances provides increased recognition of μ opioid receptors, apparently mediated by conformational changes in them. These observations support the idea that conformational changes caused by CB receptor ligands can affect the receptor partner in a heterodimer, providing therapeutic effects.

The evident analgesic effect data of the compound of the invention in the present example suggest that the compound of the invention provides similar effect to that of Hp when combined with morphine, i.e., with a lower dose of morphine in combination with Hp, the same analgesic effects. The use of the compound of the invention in combination with morphine, therefore, may contribute to lessening important implications on the addiction and tolerance that morphine causes.

### Example 13. Pharmaceutical Composition for the Treatment of Multiple Sclerosis Comprising the Compound of SEQ ID No. 1—Results In Vivo Tests

Although the exact etiology of Multiple Sclerosis is still unclear, it is speculated that auto-reactive T lymphocytes play a central role in its pathophysiology. The most common animal model of Multiple Sclerosis is experimental autoimmune encephalomyelitis (EAE), because it shares many physiological and clinical aspects. There are several models of EAE that reflect different clinical, immunological and histological aspects of human multiple sclerosis. The active induction model of EAE in mice is robust and provides replicable results and is particularly useful in the search for therapeutic agents through the use of transgenic mice challenged by autoimmune neuroinflammation. The EAE model is often used as evidence of the efficacy of novel therapeutic strategies for Multiple Sclerosis.

In this embodiment, transgenic mice knockout of the Endopeptidase 24.15 gene were challenged with MOG (Myelin Oligodendrocyte Glycoprotein), with administration of the compound of the invention SEQ ID No. 1 or saline as a control. After the introduction of the disease, a daily evaluation of the symptoms was made, according to Table 11 below.

TABLE 11
Clinical Score System

| Score | Clinical Signal | Comment |
|---|---|---|
| 0 | None | Normal walk, tail moves and can be |
| | | lifted; the tail wraps around a rounded |
| | | object if the animal is hung by the base |
| | | of the tail |
| 1 | Partially limp tail | Normal walk, fallen tail tip |
| 2 | Tail paralyzed | Normal walk, fallen tail |
| 3 | Back limb with | Uncoordinated walk, limp tail, hind |
| | paresis, | limbs respond to pinching |
| | uncoordinated | |
| | movement | |
| 4 | A paralyzed hind | Non-coordinated walk with a trailing |
| | limb | hind limb, limp tail, a hind limb does |
| | | not respond to pinching |
| 5 | Both paralyzed | Non-coordinated walk with both hind |
| | hindquarters | limbs dragging, limp tail, both hind |
| | | limbs do not respond to pinching |
| 6 | Both back limbs | Non-coordinated walk with the limbs |
| | paralyzed, | striving to pull the body, there is reflex |
| | weakness | in the front limbs after pinching, limp |
| | in the limbs | tail |
| 7 | Both hind limbs | Animal cannot move, there is reflex in |
| | paralyzed, | the fore limb after pinching, limp tail |
| | one front | |
| | limb paralyzed | |
| 8 | Both hind and | Animal cannot move, front limbs do |
| | front limbs | not respond to pinching, limp tail |
| | paralyzed | |
| 9 | Dying | No movement, breathing altered |
| 10 | Death | |

FIG. 25 shows the results of experiments conducted according to the model described above, by administration of saline (control) or SEQ ID No. 1 to knockout mice. In A, the Clinical Score curve of the Wild Type (WT) mice, that is, normal, submitted to EAE induction with MOG; In B, the Clinical Score curve of the mice knock out of the gene endopeptidase 24.15, that is, transgenic and prone to the symptoms of Multiple Sclerosis, is shown in the EAE model submitted to MOG induction; In C, the Clinical Score curve of the mice knock out 24.15, that is, transgenic and prone to the symptoms of Multiple Sclerosis in the EAE model, were submitted to induction with MOG and SEQ ID No. 1 neuroprotection. * P<0.05 24.15 KO+SEQ ID No. 1 vs WT; +P<0.05 24.15 KO vs WT. The results show an impressive reduction of clinical symptoms in animals treated with the compound of the invention, which, although being transgenic and deficient in the endopeptidase gene 24.15, exhibited behavior very close to that of normal animals. These results constitute “proof of principle” of the efficacy of this new therapeutic strategy for the curative or prophylactic treatment of Multiple Sclerosis.

It should also be noted that normal animals submitted to MOG induction have increased levels of interferon and interleukin 17; and in the knockout animals these markers—both proinflammatory—are well increased. These data, together with the response timing profile of SEQ ID No. 1-treated transgenic animals (FIG. 25), are consistent with effects on the immune response and/or anti-inflammatory effect or the compound of the invention. In this context, it is known that the cannabinoid system is involved in a variety of immune cells whose activation through receptors triggers a series of signal transductions that affect the transcription and production of cytokines and chemokines. It is known that the modulation of cannabinoid receptors inhibits migration activities of various types of immune cells. Furthermore, the influence of the cannabinoid system on immune function can occur in sites located in the periphery and also within the central nervous system, which is why the literature suggests that the cannabinoid system plays an important role in the fine regulation of homeostatic immune balance. The compound of the invention, having been shown to modulate the cannabinoid system, may interfere with this system, being potentially useful in a variety of inflammatory, immune, autoimmune conditions.

The inventive concept herein disclosed and exemplified in one or more forms was treated as an industrial secret and was not previously disclosed until the filing of this patent application or its priority. This industrial secret is immaterial asset of the applicant. The possible future publication of the patent application does not in itself constitute authorization for use by third parties, serving only as: (i) scientific knowledge to third parties of the existence of said industrial secret at the time of filing; (ii) unequivocal indication of its holder; and (iii) stimulating the development of new improvements based on the concept disclosed, in order to avoid reinvestment in the development of the same asset already held by the applicant.

It is pointed out that any commercial use requires authorization from the holder and that unauthorized use imposes penalties provided by law. In this context, it is clarified that from the disclosure of the present inventive concept, those skilled in the art may consider other forms of the invention not identical to those merely exemplified above, but that in the hypothesis of claim for commercial use such forms may be considered to be within the scope of the appended claims.

1. A compound characterized by the formula:
R₁—N-AA₁-K-AA₂-R₂
wherein:
AA₁ is an amino acid selected from the group consisting of F, W, L, I, V, P, G;
AA₂ is hydrogen or an amino acid selected from the group consisting of F, W, L, I, V, P, G;
R₁ is absent when R₂ is the amino acid L, or is hydrogen or the amino acid V; and
R₂ is absent when AA₂ is hydrogen or is hydrogen, the amino acid L when R₁ is hydrogen,
and/or modified forms thereof, cyclic-, amide- alkyl- alcoxy- halogen- hydroxyl-PEGylated forms thereof, forms modified with other functional groups, with amino acids or peptides, including non-natural amino acids, D-amino acids, its salts, and/or combinations thereof.

2. The compound according to claim 1, wherein the compound is modified, cyclized, modified with amide, alkyl, alcoxy, halogen, hydroxy, or PEG groups, or with other functional groups, with an amino acid or peptide, including non-natural ones and D-amino acids, its salts; and/or combinations thereof.

3. (canceled)

4. A method of using the peptidic compound of formula:
R₁—N-AA₁-K-AA₂-R₂
for preparing a product of pharmaceutical interest selected from a ligand for diagnostics use and a curative or prophylactic medicament for a mammal, wherein:
AA₁ is an amino acid selected from the group consisting of F, W, L, I, V, P, G;
AA₂ is hydrogen or an amino acid selected from the group consisting of F, W, L, I, V, P, G;
R₁ is absent when R₂ is the amino acid L, or is hydrogen or the amino acid V; and
R₂ is absent when AA₂ is hydrogen or is hydrogen, the amino acid L when R₁ is hydrogen,
and/or modified forms thereof, cyclic-, amide- alkyl- alcoxy- halogen- hydroxyl-PEGylated forms thereof, forms modified with other functional groups, with amino acids or peptides, including non-natural amino acids, D-amino acids, its salts, and/or combinations thereof,
comprising preparing a product of pharmaceutical interest selected from a ligand for diagnostics use and a curative or prophylactic medicament for a mammal.

5. The method according to claim 4, wherein the compound is selected from the group consisting of: NFK, NWK, NLK, SEQ ID No. 1, SEQ ID No 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, its modified forms including cyclic-, amide-, alkyl-, alcoxy-, halogen-, hydroxil-, PEG-forms, forms modified with other functional groups, or with an amino acid or peptide, including non-natural amino acids, D-amino acids, its salts; and/or combinations thereof.

6. The method according to claim 4, wherein the method comprises the preparation of a medicament for treating: disorders of energy and/or lipid metabolism; hypertension, intestinal motility disorders; the immune system; the calcium cycle equilibrium; disorders of the thyroid gland, disorders of reproductive organs, obesity, diabetes, diseases or disorders of the immune system/inflammation, osteopeny, osteoporosis, cancer.

7. The method according to claim 4, wherein the method comprises the preparation of a medicament for analgesia and/or for the treatment of migraine, pain, neuropathic pain in a mammal.

8. The method according to claim 4, wherein the method comprises the preparation of a medicament for the curative or prophylactic treatment of convulsions in a mammal.

9. The method according to claim 4, wherein the method comprises the preparation of a neuromodulator or neuroprotector medicament, a medicament for the treatment of psychiatric diseases, anxiety, squizophreny or bipolar disorder, Alzheimer, Parkinson, autism, epilepsy, multiple sclerosis.

10. (canceled)

11. (canceled)

12. (canceled)

13. (canceled)

14. (canceled)

15. (canceled)

16. (canceled)

17. (canceled)


SEQUENCE LISTING (12 sequences)

SEQ ID NO: 1 - protein, 4 aa, Artificial Sequence
  Synthesized in laboratory
NFKF

SEQ ID NO: 2 - protein, 4 aa, Artificial Sequence
  Synthesized in laboratory
NWKF

SEQ ID NO: 3 - protein, 4 aa, Artificial Sequence
  Synthesized in laboratory
NLKF

SEQ ID NO: 4 - protein, 4 aa, Artificial Sequence
  Synthesized in laboratory
NFKW

SEQ ID NO: 5 - protein, 4 aa, Artificial Sequence
  Synthesized in laboratory
NWKW

SEQ ID NO: 6 - protein, 4 aa, Artificial Sequence
  Synthesized in laboratory
NLKW

SEQ ID NO: 7 - protein, 4 aa, Artificial Sequence
  Synthesized in laboratory
NFKL

SEQ ID NO: 8 - protein, 4 aa, Artificial Sequence
  Synthesized in laboratory
NWKL

SEQ ID NO: 9 - protein, 4 aa, Artificial Sequence
  Synthesized in laboratory
NLKL

SEQ ID NO: 10 - protein, 4 aa, Artificial Sequence
  Synthesized in laboratory
VNFK

SEQ ID NO: 11 - protein, 4 aa, Artificial Sequence
  Synthesized in laboratory
VNWK

SEQ ID NO: 12 - protein, 4 aa, Artificial Sequence
  Synthesized in laboratory
VNLK